(19) United States
(12) Patent Application Publication
(10) Pub. No.: US 2010/0105617 A1
(43) Pub. Date: Apr. 29, 2010

(54) C-Terminal Attachment of Two Chemical Groups to Peptides

(75) Inventor: Bernd Peschke, Malov (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk Health Care AG, Zurich (CH)

(21) Appl. No.: 12/526,277

(22) PCT Filed: Feb. 13, 2008
(86) PCT No.: PCT/EP08/51733

§ 371 Date: Oct. 8, 2009

(30) Foreign Application Priority Data
Feb. 13, 2007 (EP) PCT/EP2007/051388
Jun. 28, 2007 (EP) 07111273.4

(51) Int. Cl. A61K 38/27; C07K 1/107; C07D 405/12; C07C 237/02

(52) U.S. Cl. 514/12; 530/345; 530/399; 548/255; 564/123

(57) ABSTRACT

The present invention relates to a method for C-terminal attachment of two property-modifying groups to a peptide.

### FIELD OF THE INVENTION

The present invention relates to the field of protein chemistry, in particular to therapeutic polypeptide conjugates.

### BACKGROUND OF THE INVENTION

The properties of peptides and proteins may be altered by attachments of chemical groups (Angew. Chem. Int. Ed. 44, 34-66 (2005)).

One possible position to attach chemical moieties is the C-terminus of the peptide or protein.

It has been shown that by utilizing the catalysis of carboxypeptidases it is possible to introduce building blocks to the C-terminus of the peptide. The building blocks may carry certain functional groups, which are not accessible on the peptide itself. These functional groups can be used in a second step as reaction handles to attach chemical moieties in order to alter the properties of the peptide. More specifically, carboxypeptidase Y has been used to introduce building blocks to peptides and hGH-derivatives (WO2005035553 (6726), WO2006084888 (7150)).

A number of pairs of chemical groups may be used as reaction handles. Such pairs react specifically with each other under suitable reaction conditions and are most conveniently chosen among chemical groups, which are not generally present in biological peptides.

One such reaction is for instance the oxime-formation reaction. Herein an alkoxylamine reacts with a carbonyl compound, such as e.g. a ketone or an aldehyde (e.g. WO2006042848 (7035)).

Another reaction is the formation of a 1,2,3-triazole by reaction of an alkyne with an azide. Commonly this reaction is performed under the catalysis of copper(I) (J. Org. Chem. 67, 3057-3064 (2002); Angew. Chem. Int. Ed. 41, 2596-2599 (2002)). There are, however, methods without the presence of a metal-catalyst (Method in Enzymology 415, 230-250 (2006)).

Another example is the Staudinger reaction. Herein an azide and an ester are reacted with each other in the presence of a triphenylphosphine derivative to form a new ester-bond (Angew. Chem. Int. Ed. 43, 3106-3116 (2004)).

In summary, so far, only methods are known to introduce a moiety with one reaction handle to the C-terminus of a peptide or protein by enzyme-catalyzed reaction. This reaction handle can be used in a second step to attach only one chemical group to the C-terminus of a peptide or protein. It may be, however, in some cases interesting to be able to attach two different moieties. For example, it could be of interest to attach both a polyethylene glycol group (PEG)-group (or another property-modifying group, which for instance could be able to increase the circulation half-life of said peptide) and a fluorescence-marker. Another example could be the aim to attach a second peptide to the C-terminus of the modified first peptide and in to attach a PEG-moiety or a fluorescence-marker to this construct of two peptides. Methods for C-terminal attachment of two chemical groups to peptides are provided by, and additional aspects, features, and advantages of the invention described in and/or will be apparent from, the description of the invention provided herein.

### SUMMARY OF THE INVENTION

The present invention provides a method of preparing a peptide, which peptide is capable of being derivatised with two property-modifying groups attached to the C-terminal of said peptide, comprising the steps of bringing a building block, which is a chemical compound comprising two or more attachment chemical groups, which attachment chemical groups are not accessible in any of the amino acid residues constituting said peptide and which attachment chemical groups are different from each other, and a incorporation chemical group, which incorporation chemical group under certain circumstances is capable of reacting with the carboxyl group in the C-terminus of the peptide, into contact with the peptide in the presence of an enzyme capable of catalysing the incorporation of said building block into the C-terminus of said peptide by catalysing a reaction between the C-terminal carboxyl group and said incorporation chemical group

The present invention provides a method of attaching two chemical moieties to the C-terminus of a peptide comprising the steps of (a) bringing a building block, which is a chemical compound comprising two or more attachment chemical groups, which attachment chemical groups are not accessible in any of the amino acid residues constituting said peptide and which attachment chemical groups are different from each other, and a incorporation chemical group, which incorporation chemical group under certain circumstances is capable of reacting with the carboxyl group in the C-terminus of the peptide, into contact with the peptide in the presence of an enzyme capable of catalysing the incorporation of said building block into the C-terminus of said peptide by catalysing a reaction between the C-terminal carboxyl group and said incorporation chemical group, (b) reacting in one or more steps a first attachment chemical group of the building block with a chemical group present on a first moiety to be attached to the peptide, which first moiety chemical group does not react with any functional groups present in the peptide and (c) reacting in one or more steps a second attachment chemical group of the building block with a chemical group present on a second moiety to be attached to the peptide, which second moiety chemical group does not react with any functional groups present on the peptide.

The present invention provides a compound produced by a method according to the invention as well as a compound obtainable by use of such a method as well as therapeutic and/or diagnostic use of some such compounds

The present invention provides a pharmaceutical composition comprising a compound produced or obtainable by a method according to the present invention.

The present invention provides a compound having the structural formula of

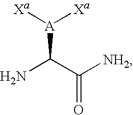

wherein A is any triradical moiety, and X^{a} and Y^{a} are chosen such that the combination of X^{a} and Y^{a}, or Y^{a} and X^{a}, are selected from a combination of ketone/1,2-diol, ketone/1,2-aminoalcohol, ketone/azide, ketone/alkyne, aldehyde/1,2-diol, aldehyde/1,2-aminoalcohol, aldehyde/azide, aldehyde/alkyne, 1,2-diol/azide, 1,2-diol/alkyne, 1,2-diol/aniline, 1,2-aminoalcohol/azide, 1,2-aminoalcohol/alkyne, 1,2-aminoalcohol/aniline, O-alkylated hydroxylamine/azide, O-alkylated hydroxylamine/alkyne, alkylated hydrazine/azide, alkylated hydrazine/alkyne, acylated hydrazine/azide, acylated hydrazine/alkyne, azide/aniline, alkyne/aniline,
a combination of ketone with a chemical group comprising

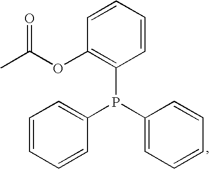

a combination of ketone with a chemical group comprising

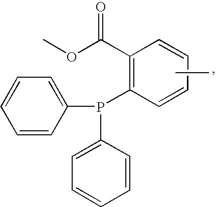

a combination of aldehyde with a chemical group comprising

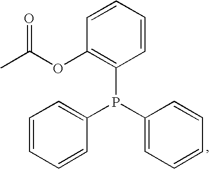

a combination of aldehyde with a chemical group comprising

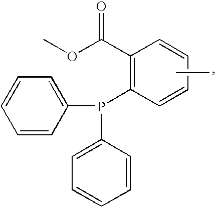

a combination of 1,2-diol with a chemical group comprising

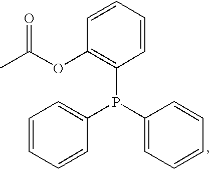

a combination of 1,2-diol with a chemical group comprising

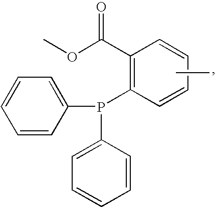

a combination of 1,2-aminoalcohol with a chemical group comprising

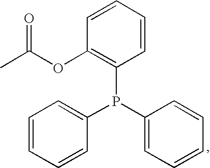

a combination of 1,2-aminoalcohol with a chemical group comprising

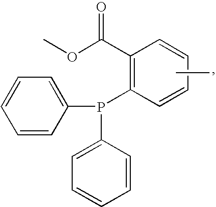

a combination of aniline with a chemical group comprising

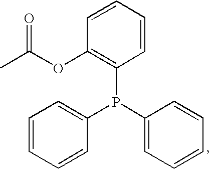

or a combination of aniline with a chemical group comprising

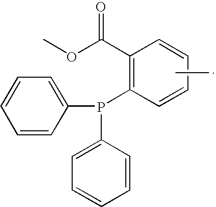

Further embodiments and a detailed description are available below.

### DETAILED DESCRIPTION OF THE INVENTION

### Method

The present invention provides a method of preparing a peptide, which peptide is capable of being derivatised with two property-modifying groups attached to the C-terminal of said peptide, comprising the step of bringing a building block, which is a chemical compound comprising two or more attachment chemical groups, which attachment chemical groups are not accessible in any of the amino acid residues constituting said peptide and which attachment chemical groups are different from each other, and a incorporation chemical group, which incorporation chemical group under certain circumstances is capable of reacting with the carboxyl group in the C-terminus of the peptide, into contact with the peptide in the presence of an enzyme capable of catalysing the incorporation of said building block into the C-terminus of said peptide by catalysing a reaction between the C-terminal carboxyl group and said incorporation chemical group.

The building block is introduced to the C-terminus of a suitable peptide by means of a reaction catalysed by a carboxypeptidase, which for the purpose of this specification shall be understood as an enzyme, which is capable of catalysing a reaction, by which the C-terminal amino acid of a peptide is replaced by a different chemical moiety.

The peptide obtained by use of such method may conveniently be derivatised further by attaching desirable moieties to the attachment chemical groups. Consequently, the present invention also provides a method of attaching two chemical moieties to the C-terminus of a peptide comprising the steps of
(a) bringing a building block, which is a chemical compound comprising two or more attachment chemical groups, which attachment chemical groups are not accessible in any of the amino acid residues constituting said peptide and which attachment chemical groups are different from each other, and a incorporation chemical group, which incorporation chemical group under certain circumstances is capable of reacting with the carboxyl group in the C-terminus of the peptide, into contact with the peptide in the presence of an enzyme capable of catalysing the incorporation of said building block into the C-terminus of said peptide by catalysing a reaction between the C-terminal carboxyl group and said incorporation chemical group,
(b) reacting in one or more steps a first attachment chemical group of the building block with a chemical group present on a first moiety to be attached to the peptide, which first moiety chemical group does not react with any functional groups present in the peptide and
(c) reacting in one or more steps a second attachment chemical group of the building block with a chemical group present on a second moiety to be attached to the peptide, which second moiety chemical group does not react with any functional groups present on the peptide.

Such a method according to the present invention contains three steps: in a first step—as also described above—the building block is introduced to the C-terminus of a suitable peptide by means of a reaction catalysed by a carboxypeptidase, which for the purpose of this specification shall be understood as an enzyme, which is capable of catalysing a reaction, by which the C-terminal amino acid of a peptide is replaced by a different chemical moiety. In a second and a third step two moieties may be attached stepwise to the C-terminus of the peptide or protein via the orthogonal reaction handles X^{a} and Y^{a} in any convenient order.

The enzyme catalyzed modification of the C-termini of the peptide may be performed by a variety of carboxypeptidases. A carboxypeptidase is an enzyme, which is capable of catalysing a reaction, by which the C-terminal amino acid of a peptide is replaced by a different chemical moiety. A variant of such carboxypeptidase, which retains the ability to catalyse a reaction, by which the C-terminal amino acid of a peptide is replaced by a different chemical moiety (a “functional” variant or fragment), is also useful in said method as a fragments of such carboxypeptidases or carboxypeptidase variants. In one embodiment, a serine-type carboxypeptidase or cystein-type carboxypeptidase (or a functional variant and/or fragment of one of those) is used as such a carboxypeptidase. For the purpose of this specification, a serine-carboxypeptidase is to be understood as those carboxypeptidases which are classified in the E.C. system of the Nomenclature Committee of the International Union of Biochemistry and Molecular Biology (NC-IUBMB) under the group of E.C.3.4.16 or functional variants and/or fragments thereof. For the purpose of this specification, cystein-type carboxypeptidase is to be understood as those carboxypeptidases which are classified in the E.C. system of the Nomenclature Committee of the International Union of Biochemistry and Molecular Biology (NC-IUBMB) under the group of E.C.3.4.18 or functional variants and/or fragments thereof. In one embodiment, a serine carboxypeptidase or a functional variant and/or fragment thereof. is used. In a further embodiment, said serine carboxypeptidase is carboxypeptidase Y (CPY). In one embodiment the enzyme is a variant or a fragment of carboxypeptidase Y (or of a functional variant thereof), which variant and/or fragment retains the ability to catalyse a reaction, by which the C-terminal amino acid of a peptide is replaced by a different chemical moiety. Several variants of carboxypeptidase Y are known in the art; see for instance WO9838285. Functional derivatives of all said carboxypeptidases may also conveniently be used.

The term “peptide” or “polypeptide” (the two terms are used interchangeably herein) as used herein means a compound comprising at least five constituent amino acid residues covalently connected by peptide bonds. In one embodiment, the peptide comprises at least one chain comprising at least about 10, such as at least about 15, for instance at least about 20, such as at least about 30, for instance at least about 40, such as at least about 50, for instance at least about 75, such at least about 100 amino acid residues covalently connected to each other by peptide bonds.

The constituent amino acids may be from the group of the amino acids encoded by the genetic code and they may be natural amino acids which are not encoded by the genetic code, as well as synthetic amino acids. Natural amino acids which are not encoded by the genetic code are e.g. hydroxyproline, y-carboxy-glutamic acid, ornithine, phophoserine, D-alanine, D-glutamic acid. Synthetic amino acids comprise amino acids manufactured by organic synthesis, e.g. D-isomers of the amino acids encoded by the genetic code and Aib (α-aminoisobutyric acid), Abu (α-aminobutyric acid), Tle (tert-butylglycine), and β-alanine. A peptide may comprise a single peptide chain or it may comprise more than one peptide chain, such as human growth hormone being a single chain and human insulin being two chains connected by disulphide bonds.

The term “variant” as used herein refers either to a naturally occurring variation of a given polypeptide or a recombinantly prepared or otherwise modified variation of a given peptide or protein in which one or more amino acid residues have been modified by amino acid substitution, addition, deletion, insertion or invertion.

The term “derivative” as used herein refers to a polypeptide or variant or fragment thereof which is modified, i.e., by covalent attachment of any type of molecule, preferably having bioactivity, to the parent polypeptide. Typical modifications are amides, carbohydrates, alkyl groups, acyl groups, esters, PEGylations and the like. This should be taken to mean that the polypeptide compounds according to the present invention may be already (or previously) conjugated to a third chemical group, e.g. a non-polypeptide moiety. Hence, in one embodiment, a method according to the present invention also comprises conjugating an already (or previously) conjugated polypeptide to two further chemical groups. Such previous conjugation may for instance have been performed via a reduced cysteine residue, or it may be performed via a glutamic acid residue, such as described in WO2002077218 (6286) and WO200158935 or as it is otherwise known in the art.

### Compounds

The present invention also provides compounds, or building blocks, useful for use in a method according to the present invention. Such building blocks may conveniently have the general structural formula of

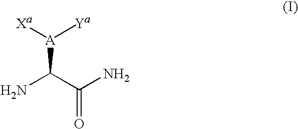

in which A is any triradical moiety, which can be used as a scaffold for the functional attachment groups X^{a} and Y^{a}. For the purpose of the present specification, such compounds will also be named as “building blocks”. X^{a} is a chemical group, which can be used as reaction handle to attach a chemical moiety via a chemical reaction and Y^{a} is also a chemical group, which can be used as a reaction handle to attach a chemical moiety via a chemical reaction. The choice of which of the chemical groups is to be seen as “X^{a}” and which is to be seen as “Y^{a}” is of course arbitrary, and does therefore not propose any limitation on the scope of the present invention. X^{a} and Y^{a} are chosen orthogonally so that it is possible to perform a reaction with only one of the functional groups. Thereafter it is possible to perform a reaction with the other functional group. Examples for two orthogonal groups of reaction handles could be for example the group of aldehyde, ketone, and O-alkylated hydroxylamine for X^{a} (or Y^{a}) and the group of e.g. azide and alkyne on the other hand for Y^{a} (or X^{a}).

In one embodiment, the attachment groups, X^{a} and Y^{a}, are selected from the group consisting of ketones, aldehydes, 1,2-diols, 1,2-aminoalcohols, O-alkylated hydroxylamines, alkylated hydrazines, acylated hydrazines, azides, alkynes, anilines or comprise a group selected from

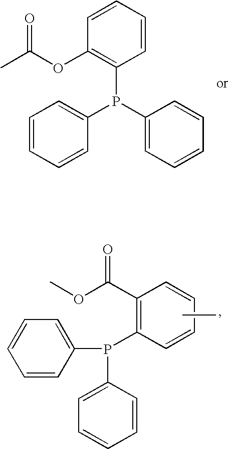

wherein the two attachment groups are different from each other.

In one embodiment, the combination of X^{a} and Y^{a} are chosen such that the combination of X^{a} and Y^{a}, or Y^{a} and X^{a}, are selected from a combination of ketone/1,2-diol, ketone/1,2-aminoalcohol, ketone/azide, ketone/alkyne, aldehyde/1,2-diol, aldehyde/1,2-aminoalcohol, aldehyde/azide, aldehyde/alkyne, 1,2-diol/azide, 1,2-diol/alkyne, 1,2-diol/aniline, 1,2-aminoalcohol/azide, 1,2-aminoalcohol/alkyne, 1,2-aminoalcohol/aniline, O-alkylated hydroxylamine/azide, O-alkylated hydroxylamine/alkyne, alkylated hydrazine/azide, alkylated hydrazine/alkyne, acylated hydrazine/azide, acylated hydrazine/alkyne, azide/aniline, alkyne/aniline, or a combination of ketone with one of the groups of

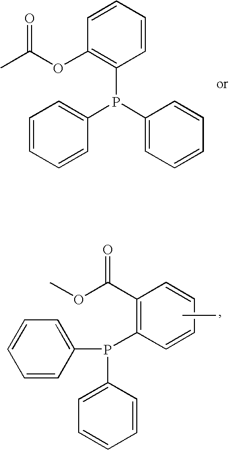

a combination of aldehyde with one of the groups of

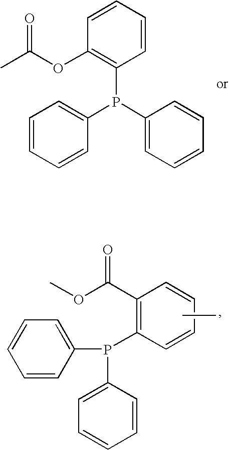

a combination of 1,2-diol with one of the groups of

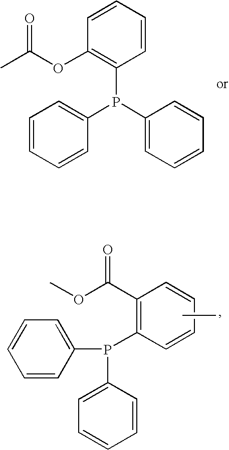

a combination of 1,2-aminoalcohol with one of the groups of

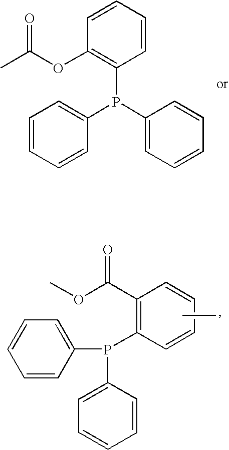

or a combination of aniline with one of the groups of

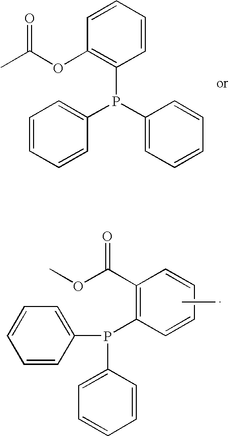

In one embodiment, the building block comprises a combination of two attachment chemical groups selected from the list of: ketone/azide or ketone/alkyne.

The incorporation chemical group should be selected so that it can react with the C-terminal of the peptide, while not reacting to any significant extent with other amino acid residues of the peptide, nor with the attachment chemical groups. The choice of incorporation chemical group may depend on the nature of the C-terminal amino acid residue. In one embodiment, the C-terminal amino acid of said peptide to be derivatised is selected from the group consisting of isoleucine, phenylalanine, leucine, tryptophan, alanine methionine, valine, and glycine.

In one embodiment, the incorporation chemical group is an amine. In one embodiment, and the attachment chemical groups comprise a combination of two chemical groups selected from ketone/1,2-diol, ketone/1,2-aminoalcohol, ketone/azide, ketone/alkyne, aldehyde/1,2-diol, aldehyde/1,2-aminoalcohol, aldehyde/azide, aldehyde/alkyne, 1,2-diol/azide, 1,2-diol/alkyne, 1,2-diol/aniline, 1,2-aminoalcohol/azide, 1,2-aminoalcohol/alkyne, 1,2-aminoalcohol/aniline, O-alkylated hydroxylamine/azide, O-alkylated hydroxylamine/alkyne, alkylated hydrazine/azide, alkylated hydrazine/alkyne, acylated hydrazine/azide, acylated hydrazine/alkyne, azide/aniline, alkyne/aniline, a combination of ketone with a chemical group comprising

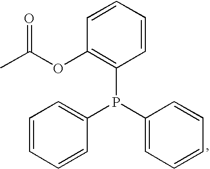

a combination of ketone with a chemical group comprising

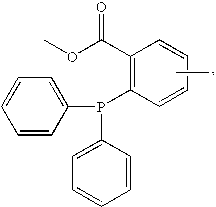

a combination of aldehyde with a chemical group comprising

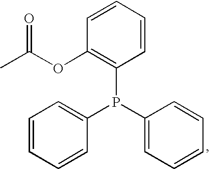

a combination of aldehyde with a chemical group comprising

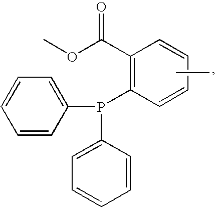

a combination of 1,2-diol with a chemical group comprising

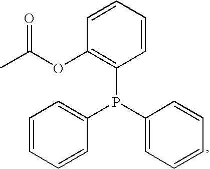

a combination of 1,2-diol with a chemical group comprising

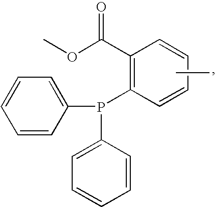

a combination of 1,2-aminoalcohol with a chemical group comprising

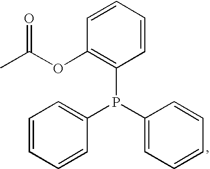

a combination of 1,2-aminoalcohol with a chemical group comprising

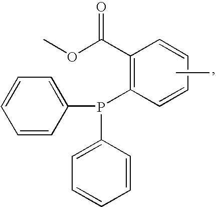

a combination of aniline with a chemical group comprising

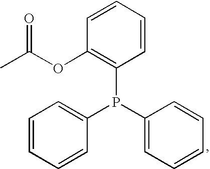

or a combination of aniline with a chemical group comprising

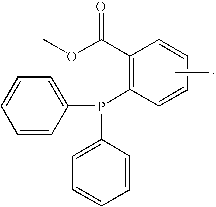

In one embodiment, the incorporation chemical group is an alpha amino amide. In one embodiment, the incorporation chemical group is an alpha-amino amide and the attachment chemical groups comprise a combination of ketone/azide or ketone/alkyne.

The triradical moiety A may be any triradical moiety with a molecular weight not exceeding 750 Da. To ensure water-solubility of a compound of formula (I), the triradical moiety A is chosen in a way, that a compound of formula (I) has a log D<6 at least one pH between pH 3 and 11. Log D is the decadic logarithm of the distribution coefficient of a compound of formula (I) in a octanol/water system, wherein the distribution coefficient is the ratio of the sum of the concentrations of all forms of the compound (ionized plus unionized) in each of the two phases.

In one embodiment, the triradical moiety A comprises a triradical of the formula

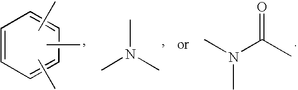

In one embodiment, the triradical moiety A comprises a triradical of the formula

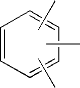

In both of the above embodiments, the triradical moiety A may also comprise further chemical groups attached to the stated triradical moieties and to which further chemical groups the Xa, Ya and

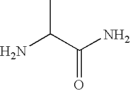

groups are attached as shown in the examples herein.

As it is readily apparent from the description of the invention, it is to be understood though, that it is not the intention that the invention should be limited in the choice of the triradical moiety A.

In one embodiment, the building block has the structural formula of

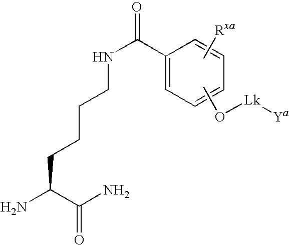

wherein Lk is a biradical of an alkane, alkene, arene, or heteroarene group, and R^{xa} is a chemical group comprising the attachment group X^{a}. In one embodiment, Lk is a biradical of an alkane.

In one embodiment, the building block has the structural formula of

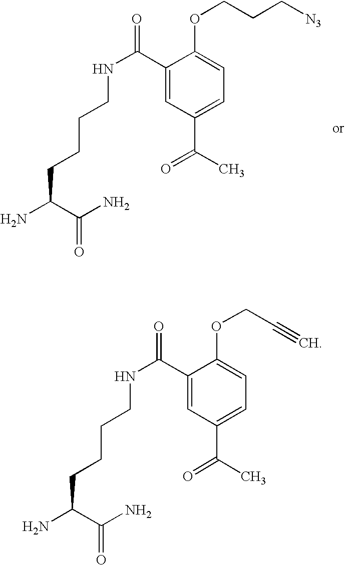

As it is readily apparent, the invention is independent of which chemical moieties are to be attached to the chemical groups X^{a} and Y^{a} as long as these are chosen in a form so that they are able to participate in a chemical reaction with X^{a} and Y^{a} respectively as described elsewhere herein. The structure and/or nature of the property-modifying groups, which is attached using a method according to the present invention, are determined by the need of the person wanting to attach such two moieties. By way of example only, such property-modifying groups may be radicals of peptides, polymers, fluorescence-tags, reporter groups, or affinity tags, such as for instance a PEG-moiety or a biotin-carrying moiety.

The present invention also provides a compound produced by a method according to the invention as well as compounds obtainable by use of a method according to the invention.

For the purpose of this specification, the term “cyclic” in relations to radicals and other chemical groups or molecules encompasses mixed cyclic/linear molecules, that is molecules with both a linear part and a cyclic part.

The term “alkane” is intended to encompass linear or branched saturated hydrocarbons. In one embodiment, an alkane is a “C₁₋₁₀alkane”, which is intended to encompass linear or branched saturated hydrocarbons having from 1 to 10 carbon atoms. Particular examples are methane, ethane, n-propane, isopropane, n-butane, isobutane, sec-butane, tert-butane, n-pentane, isopentane, n-octane, etc.

The term “alkyl” is intended to encompass a monoradical of an alkane. In one embodiment, an alkyl is a C₁₋₁₀alkyl. A “C₁₋₁₀alkyl” is a monoradical of an C₁₋₁₀alkane.

The term “alkene” is intended to encompass olefinically unsaturated branched or straight hydrocarbon groups having at least one double bond. In one embodiment, an alkene is a C₂₋₁₀alkene. The term “C₂₋₁₀alkene” is intended to encompass olefinically unsaturated branched or straight hydrocarbon groups having from 2 to 10 carbon atoms and at least one double bond. Examples of such groups include, but are not limited to, ethylene, 1-propene, 2-propene, isopropene, 1,3-butadiene, 1-butene, hexene, pentene and the like.

The term “alkenyl” is intended to encompass a monoradical of an alkene. In one embodiment, an alkenyl is a C₂₋₁₀alkenyl. The term “C₂₋₁₀alkenyl” is intended to encompass a monoradical of a C₂₋₁₀alkene.

The term “alkyne” is intended to encompass unsaturated branched or straight hydrocarbon groups having at least one triple bond. The term “C₂₋₁₀alkyne” is intended to encompass unsaturated branched or straight hydrocarbon groups having from 2 to 10 carbon atoms and at least one triple bond. Examples of such groups include, but are not limited to, 1-propyne, 2-propyne 1-butyn, 2-butyne, 1-pentyne, 2-pentyne and the like.

The term “alkynyl” is intended to encompass a monoradical of an alkyne. In one embodiment, an alkynyl is a C₂₋₁₀alkynyl. The term “C₂₋₁₀alkynyl” is intended to encompass a monoradical of a C₂₋₁₀alkyne.

The term “arene” is intended to encompass a compound 6 membered monycyclic aromatic system or a 10 membered bicyclic aromatic system such as benzene or naphthalene.

The term “heteroarene” is intended to encompass a compound comprising a 5-6 membered monocyclic aromatic system or a 9-10 membered bicyclic aromatic system containing one or more heteroatoms (such as for instance nitrogen, oxygen, phosphorous or sulfur), such as furan, thiophene, pyrrole, imidazole, pyrazole, triazole, pyridine, pyrazine, pyrimidine, pyridazine, isothiazole, isoxazole, oxazole, oxadiazole, thiadiazole, quinoline, isoquinoline, quinazoline, quinoxaline, indole, benzimidazole, benzofuran, pteridine and purine and the like.

The term “heteroaryl” is intended to encompass a monoradical of a heteroarene.

The term “heteroalkane” is intended to encompass straight or branched saturated carbon chains, wherein one or more carbon atoms are substituted by a heteroatom (such as for instance nitrogen, oxygen, phosphorous or sulfur). In one embodiment, a heteroalkane is a C₁₋₁₀heteroalkane. The term “C₁₋₁₀heteroalkane” is intended to encompass heteroalkanes containing from 1 to 10 carbon atoms wherein one or more carbon atoms are substituted by a heteroatom (such as for instance nitrogen, oxygen, phosphorous or sulfur). Examples of such heteroalkanes are for instance

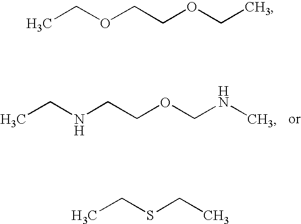

The term “heteroalkyl” is intended to encompass a monoradical of a heteroalkane.

In one embodiment, a heteroalkyl is a C₁₋₁₀heteroalkyl. The term “C₁₋₁₀heteroalkyl” is intended to encompass a monoradical of a C₁₋₁₀heteroalkane.

The term “heteroalkene” is intended to encompass olefinically unsaturated branched or straight hydrocarbon groups having at least one double bond, wherein one or more carbon atoms are substituted by a heteroatom (such as for instance nitrogen, oxygen, phosphorous or sulfur). In one embodiment, a heteroalkene is a C₂₋₁₀heteroalkene. The term “C₂₋₁₀heteroalkene” is intended to encompass heteroalkenes having from 2 to 10 carbon atoms and at least one double bond, wherein one or more carbon atoms are substituted by a heteroatom (such as for instance nitrogen, oxygen, phosphorous or sulfur), such as

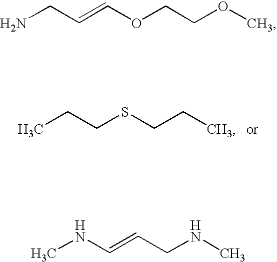

The term “heteroalkenyl” is intended to encompass a monoradical of a C₂₋₁₀heteroalkene. In one embodiment, a heteroalkenyl is a C₂₋₁₀heteroalkenyl. The term “C₂₋₁₀heteroalkenyl” is intended to encompass a monoradical of a C₂₋₁₀heteroalkene.

The terms “heteroalkyne” is intended to encompass unsaturated branched or straight hydrocarbon groups having at least one triple bond, wherein one or more carbon atoms are substituted by a heteroatom (such as for instance nitrogen, oxygen, phosphorous or sulfur). In one embodiment, a heteroalkyne is a C₂₋₁₀heteroalkyne. The term “C₂₋₁₀heteroalkyne” is intended to encompass heteroalkynes having from 2 to 10 carbon atoms and at least one triple bond, wherein one or more carbon atoms are substituted by a heteroatom (such as for instance nitrogen, oxygen, phosphorous or sulfur), such as

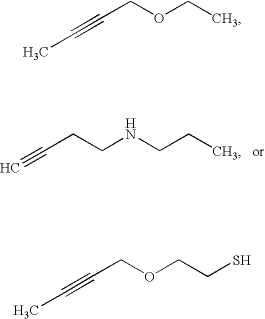

The term “heteroalkynyl” is intended to encompass a monoradical of an heteroalkyne. In one embodiment, heteroalkynyl is a C₂₋₁₀heteroalkynyl. The term “C₂₋₁₀heteroalkynyl” is intended to encompass a monoradical of a C₂₋₁₀heteroalkyne.

### Pharmaceutical Use

In one embodiment of a method according to the present invention, the peptide to be derivatised is useful in therapy or diagnosis of a patient. As described earlier, depending on the therapeutic peptide in question, it may be particularly interesting to be able to derivatise such peptides with two C-terminal attachment groups. A host of therapeutic peptides are known in the art and the invention is not to be limited by the choice of peptide. However, for exemplary purposes, the peptide may be for instance human growth hormone as described in for instance WO2005/035553. In one embodiment, the peptide is a human growth hormone or a variant thereof, derivatives thereof, which variants, derivatives, or derivatives of variants have retain the therapeutic relevant functions of human growth hormone, or fragments thereof, which fragments have retain the therapeutic relevant functions of human growth hormone. In one embodiment, the peptide is hGH-Leu-Ala. Such compounds may be useful in the treatment of diseases or disorders, which may be treated with growth hormone, for instance growth hormone deficiency (GHD), e.g. in adults; Turner syndrome; Prader-Willi syndrome (PWS); Noonan syndrome; Downs syndrome; chronic renal disease; juvenile rheumatoid arthritis; cystic fibrosis; HIV-infection in children receiving HAART treatment (HIV/HALS children); children born small for gestational age (SGA); short stature in children born with very low birth weight (VLBW); skeletal dysplasia; hypochondroplasia; achondroplasia; idiopathic short stature (ISS); fractures in or of long bones, such as tibia, fibula, femur, humerus, radius, ulna, clavicula, metacarpea, metatarsea, and digit; fractures in or of spongious bones, such as the skull, base of hand and base of foot; post-surgical treatment of patients who have undergone tendon or ligament surgery in, e.g., the hand, knee, or shoulder; treatment of patients undergoing distraction osteogenesis; post-surgical treatment of patients who have undergone hip or discus replacement, meniscus repair, spinal fusions or prosthesis fixation, such as in the knee, hip, shoulder, elbow, wrist or jaw; treatment of patients who have undergone fixation of osteosynthesis material, such as nails, screws or plates; non-union or mal-union of fractures; treatment of patients after osteatomia, e.g. from the tibia or 1st toe; treatment of patients after graft implantation; articular cartilage degeneration in the knee caused by trauma or arthritis; osteoporosis in patients with Turner syndrome; osteoporosis in men; treatment of adult patients in chronic dialysis (APCD); treatment of elderly patients in chronic dialysis; cardiovascular disease in APCD; cachexia in APCD; cancer in APCD; chronic obstructive pulmonary disease in APCD; HIV in APCD; chronic liver disease in APCD; fatigue syndrome in APCD; Crohn's disease; impaired liver function; treatment of males with HIV infections; short bowel syndrome; central obesity; HIV-associated lipodystrophy syndrome (HALS); male infertility; treatment of patients after major elective surgery; treatment of patients with negative nitrogen balance; alcohol/drug detoxification or neurological trauma; aging; frail elderly; osteoarthritis; treatment of patients with traumatically damaged cartilage; erectile dysfunction; fibromyalgia; memory disorders; depression; traumatic brain injury; subarachnoid haemorrhage; very low birth weight; metabolic syndrome; glucocorticoid myopathy; and short stature due to glucucorticoid treatment in children.

In one embodiment, the present invention provides a pharmaceutical composition comprising a compound produced by or obtainable by use of a method according to the present invention and a pharmaceutically acceptable carrier or excipient. The invention further provides the use of such compounds treating or diagnosing a disorder or disease of a patient, which disease or disorder is dependent on the therapeutic use of said peptide as well as the use of such compounds for the manufacture of a pharmaceutical composition for the treatment and/or diagnosis of a disorder or disease of a patient, which disease or disorder is dependent on the therapeutic use of said peptide.

The present invention also provides a method for the production of a pharmaceutical composition useful for treatment and/or diagnosis of a disorder or disease of a patient, which method comprises
a) obtaining a peptide useful in the treatment and/or diagnosis of said disease or disorder,
b) derivatising said peptide according to a method according to the invention as described above, and
c) formulating said derivatised peptide into a pharmaceutically acceptable composition.

Such formulation and/or purification work needed for bringing the derivatised peptide into a pharmaceutically acceptable composition is within the skill of a person skilled in the art. The compounds may thus be formulated into pharmaceutical compositions comprising the compounds and a pharmaceutically acceptable carrier or diluent. Such carriers include water, physiological saline, ethanol, polyols, e.g., glycerol or propylene glycol, or vegetable oils. As used herein, “pharmaceutically acceptable carriers” also encompasses any and all solvents, dispersion media, coatings, antifungal agents, preservatives, isotonic agents and the like. Except insofar as any conventional medium is incompatible with the active ingredient and its intended use, its use in the compositions of the present invention is contemplated.

The compositions may be prepared by conventional techniques and appear in conventional forms, for example, capsules, tablets, solutions or suspensions. The pharmaceutical carrier employed may be a conventional solid or liquid carrier. Examples of solid carriers are lactose, terra alba, sucrose, talc, gelatine, agar, pectin, acacia, magnesium stearate and stearic acid. Examples of liquid carriers are syrup, peanut oil, olive oil and water. Similarly, the carrier or diluent may include any time delay material known to the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

The pharmaceutical compositions can be sterilised and mixed, if desired, with auxiliary agents, emulsifiers, salt for influencing osmotic pressure, buffers and/or colouring substances and the like, which do not deleteriously react with the active compounds.

The route of administration may be any route, which effectively transports the active compound to the appropriate or desired site of action, such as oral or parenteral, e.g., rectal, transdermal, subcutaneous, intranasal, intramuscular, topical, intravenous, intraurethral, ophthalmic solution or an ointment, the oral route being preferred.

If a solid carrier for oral administration is used, the preparation can be tabletted, placed in a hard gelatine capsule in powder or pellet form or it can be in the form of a troche or lozenge. The amount of solid carrier may vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

For nasal administration, the preparation may contain a compound of formula (I) dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet, which may be prepared by conventional tabletting techniques, contains

| | | |
|---|---|---|
| Core: | | |
| Active compound (as free compound or salt thereof) | 10 | mg |
| Colloidal silicon dioxide (Areosil ®) | 1.5 | mg |
| Cellulose, microcryst. (Avicel ®) | 70 | mg |
| Modified cellulose gum (Ac-Di-Sol ®) | 7.5 | mg |
| Magnesium stearate | | |
| Coating: | | |
| HPMC | approx. 9 | mg |
| *Mywacett ® 9-40 T | approx. 0.9 | mg |

*Acylated monoglyceride used as plasticizer for film coating.

The compounds of the invention may be administered to a mammal, especially a human in need of such treatment, prevention, elimination, alleviation or amelioration of various diseases or disorders. Such mammals also include animals, both domestic animals, e.g. household pets, and non-domestic animals such as wildlife.

Usually, dosage forms suitable for oral, nasal, pulmonal or transdermal administration comprise from about 0.001 mg to about 100 mg, preferably from about 0.01 mg to about 50 mg of the compounds of formula I admixed with a pharmaceutically acceptable carrier or diluent.

The compounds may be administered concurrently, simultaneously, or together with a pharmaceutically acceptable carrier or diluent, whether by oral, rectal, or parenteral (including subcutaneous) route. The compounds are often, and preferably, in the form of an alkali metal or earth alkali metal salt thereof.

Suitable dosage ranges varies as indicated above depending upon the exact mode of administration, form in which administered, the indication towards which the administration is directed, the subject involved and the body weight of the subject involved, and the preference and experience of the physician or veterinarian in charge.

General method A

### Preparation of a Building Block:

A compound of formula E1, which is a compound of the general formula (I), may be prepared from an ester of general formula A1, which may be alkylated at its free phenolic group with a suitable alkylation reagent, LG-Lk-Y^{a′}, in which LG is a suitable leaving group such as e.g. bromine, mesylate or tosylate, Lk is a suitable linker for the group Y^{a}, and Y^{a′} is a group which is either Y^{a} or a group which can be transformed into Y^{a}. R^{xa} is a residue, containing a functional group X^{a}. Both X^{a} and Y^{a} may be suitably protected. The compound B1 may be saponificated by e.g. treatment with aqueous base to give acid C1, which upon activation and reaction with a suitably protected aminoamide may yield a compound of the general formula D1. The compound of the general formula D1 may be deprotected under suitable conditions, giving rise to the compound E1.

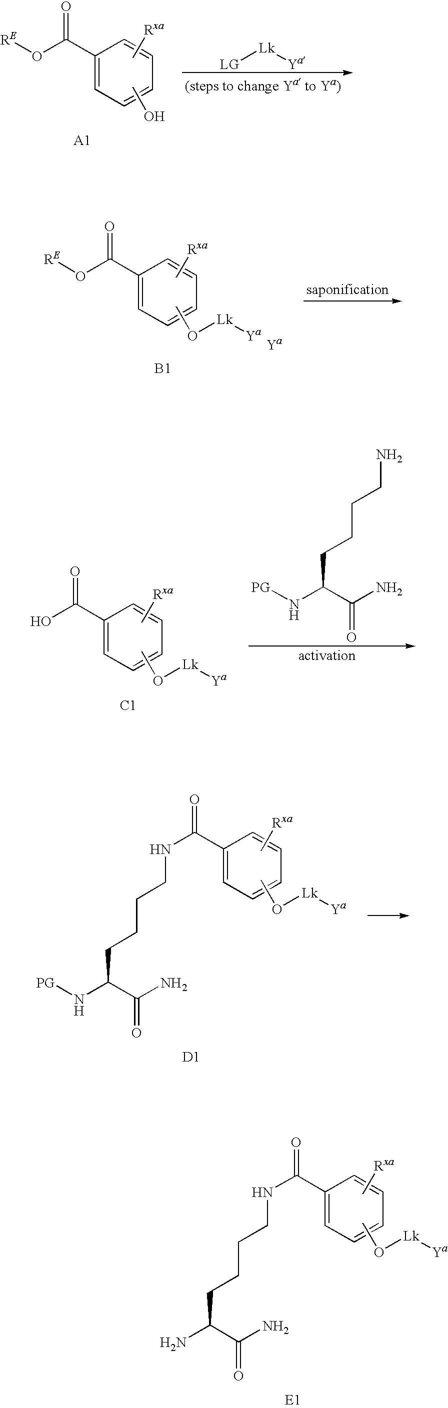

Enzyme-Catalyzed Introduction into the Protein or Peptide

The compound of formula E1 may be reacted with a suitable peptide, in which R^{AA} is the amino acid residue of the first amino acid in the peptide, in the presence of a suitable enzyme for example CPY to yield a peptide F1, in which the first amino acid of the original peptide is exchanged with the building block E1.

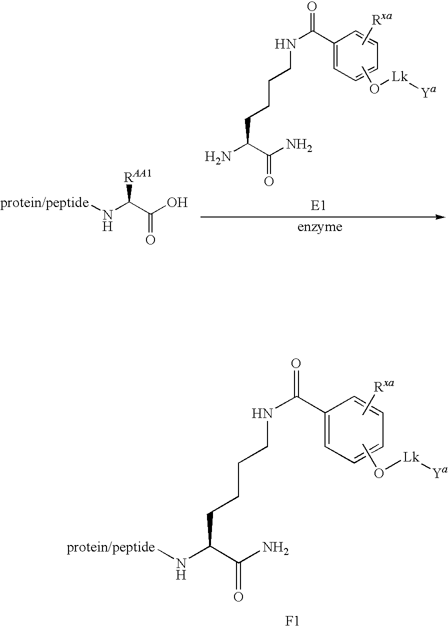

### Attachment of the First Moiety

The group Y^{a} in the compound of the formula F1 may be reacted with a functional group Y^{b}. The functional group Y^{b} is contained in a residue R^{yb} which is attached to a moiety 1. It is desired to attach moiety 1 to the peptide. The functional group Y^{b} is capable of reacting with the functional group Y^{a}. The reaction of Y^{a} with the functional group of R^{yb} may form a linking moiety R^{y-y} in compound of the formula G1. Y^{a} and Y^{b} are chosen from functional groups, which are not elsewhere accessible in the protein or peptide F1 and which do not react with other functional groups accessible in the protein or peptide F1.

For example, Y^{a} may for example be alkyne and Y^{b} may be azide or vice versa. In another example, Y^{a} may for example be chosen from the group of ketone or aldehyde and Y^{b} may be an O-alkoxylamine or vice versa. Other possibilities for Y^{a} and Y^{b} are described elsewhere herein.

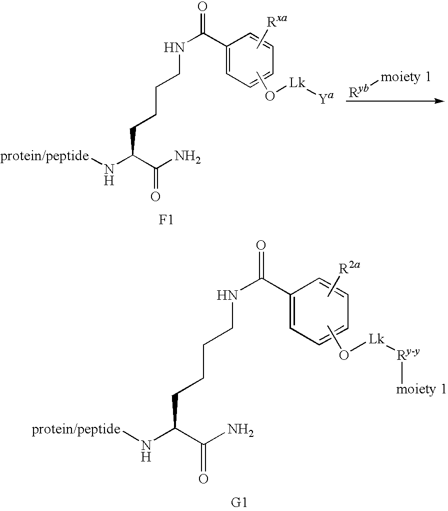

### Attachment of the Second Moiety

The group X^{a} which is contained in the residue R^{xa} in the compound of the formula G1 may be reacted with a group X^{b}. The functional group X^{b} is contained in the residue R^{xb}, which is attached to a moiety 2 It is desired to attach moiety 2 to the peptide. The reaction of the functional groups X^{a} with X^{b} may form a linking moiety R^{x-x} in compound of the formula H1. X^{a} and X^{b} are chosen from functional groups, which are not elsewhere accessible in the peptide G1 and which do not react with other functional groups accessible in the peptide G1.

For example, if r was chosen from the group of e.g. ketone, aldehyde or an O-alkoxylamine, X^{a} may for example be alkyne and X^{b} may be azide or vice versa. In another example, if r is chosen from the group of alkyne or azide, X^{a} may be chosen for example from the group of ketone or aldehyde and X^{b} may be an O-alkoxylamine. Other possibilities for X^{a} and X^{b} are described elsewhere herein.

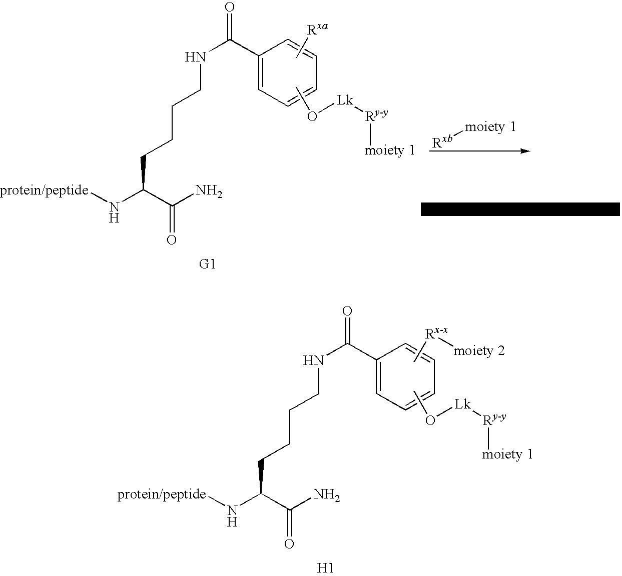

### General Method B

### Alternative Order of Attachment of Moieties

### Attachment of the First Moiety

The residue R^{xa} contains a functional group X^{a}. The group X^{a} in the compound of the formula F1 may be reacted with a functional group X^{b}. The functional group X^{b} is contained in a residue X^{b} which is attached to a moiety 1. It is desired to attach moiety 1 to the peptide. The functional group X^{b} is capable of reacting with the functional group X^{a}. The reaction of X^{a} with the functional group of R^{xb} may form a linking moiety R^{x-x} in compound of the formula G1. X^{a} and X^{b} are chosen from functional groups, which are not elsewhere accessible in the peptide F1 and which do not react with other functional groups accessible in the peptide F1.

For example X^{a} may for example be alkyne and X^{b} may be azide or vice versa. In another example X^{a} may for example be chosen from the group of ketone or aldehyde and X^{b} may be an O-alkoxylamine or vice versa. Other possibilities for X^{a} and X^{b} are described elsewhere herein.

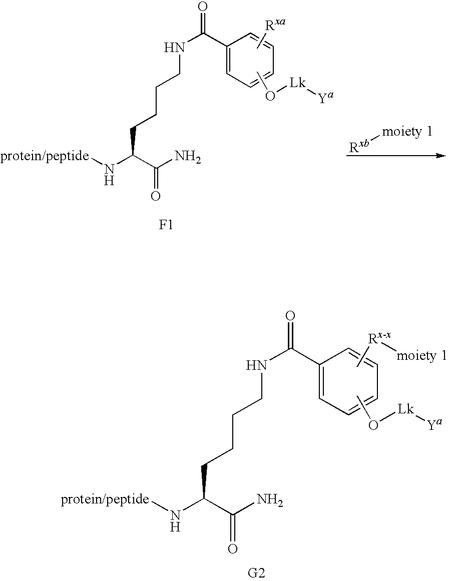

### Attachment of the Second Moiety

The functional group Y^{a} in the compound of the formula G2 may be reacted with a functional group Y^{b}. Y^{b} is contained in a residue R^{yb} which is attached to a moiety 2. It is desired to attach moiety 2 to the peptide. The reaction of Y^{a} with the functional group in R^{yb} may form a linking moiety R^{y-y} in compound of the formula H2. Y^{a} and Y^{b} are chosen from functional groups, which are not elsewhere accessible in the peptide G2 and which do not react with other functional groups accessible in the peptide G2.

For example if X^{a} was chosen from the group of e.g. ketone, aldehyde or an O-alkoxylamine, Y^{a} may for example be alkyne and Y^{b} may be azide or vice versa. In another example, if X^{a} was chosen from the group of alkyne or azide, Y^{a} may be chosen for example from the group of ketone or aldehyde and Y^{b} may be an O-alkoxylamine. Other possibilities for Y^{a} and Y^{b} are described elsewhere herein.

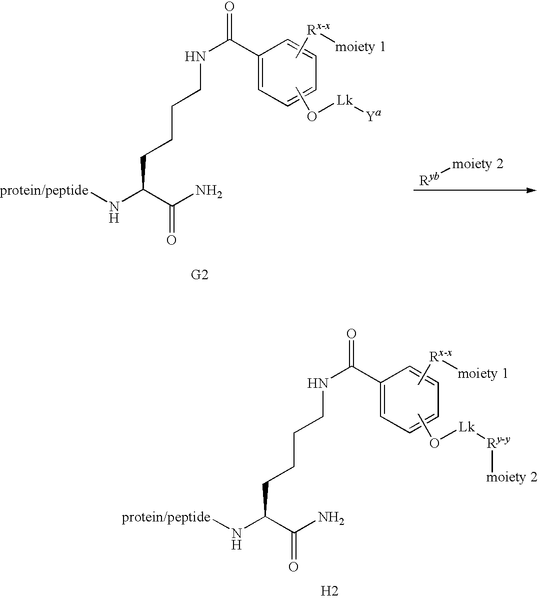

The following is a list of embodiments of the present invention.

### Embodiment 1

A method of preparing a peptide, which peptide is capable of being derivatised with two property-modifying groups attached to the C-terminal of said peptide, comprising the step of
(a) bringing a building block, which is a chemical compound comprising two or more attachment chemical groups, which attachment chemical groups are not accessible in any of the amino acid residues constituting said peptide and which attachment chemical groups are different from each other, and a incorporation chemical group, which incorporation chemical group under certain circumstances is capable of reacting with the carboxyl group in the C-terminus of the peptide, into contact with the peptide in the presence of an enzyme capable of catalysing the incorporation of said building block into the C-terminus of said peptide by catalysing a reaction between the C-terminal carboxyl group and said incorporation chemical group.

### Embodiment 2

A method of attaching two chemical moieties to the C-terminus of a peptide comprising the steps of
(a) bringing a building block, which is a chemical compound comprising two or more attachment chemical groups, which attachment chemical groups are not accessible in any of the amino acid residues constituting said peptide and which attachment chemical groups are different from each other, and a incorporation chemical group, which incorporation chemical group under certain circumstances is capable of reacting with the carboxyl group in the C-terminus of the peptide, into contact with the peptide in the presence of an enzyme capable of catalysing the incorporation of said building block into the C-terminus of said peptide by catalysing a reaction between the C-terminal carboxyl group and said incorporation chemical group,
(b) reacting in one or more steps a first attachment chemical group of the building block with a chemical group present on a first moiety to be attached to the peptide, which first moiety chemical group does not react with any functional groups present in the peptide and
(c) reacting in one or more steps a second attachment chemical group of the building block with a chemical group present on a second moiety to be attached to the peptide, which second moiety chemical group does not react with any functional groups present on the peptide.

### Embodiment 3

A method according to embodiment 1 or embodiment 2, wherein the attachment groups of the building block are selected from the group consisting of ketones, aldehydes, 1,2-diols, 1,2-aminoalcohols, O-alkylated hydroxylamines, alkylated hydrazines, acylated hydrazines, azides, alkynes, anilines or comprise a group selected from

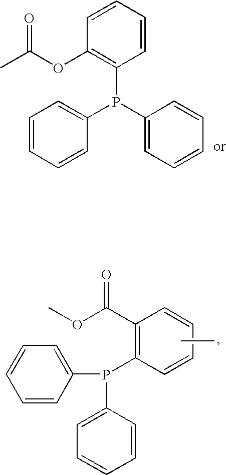

wherein the two attachment groups are different from each other.

### Embodiment 4

A method according to any of embodiments 1 to 3, wherein the building block comprises a combination of two attachment chemical groups selected from ketone/1,2-diol, ketone/1,2-aminoalcohol, ketone/azide, ketone/alkyne, aldehyde/1,2-diol, aldehyde/1,2-aminoalcohol, aldehyde/azide, aldehyde/alkyne, 1,2-diol/azide, 1,2-diol/alkyne, 1,2-diol/aniline, 1,2-aminoalcohol/azide, 1,2-aminoalcohol/alkyne, 1,2-aminoalcohol/aniline, O-alkylated hydroxylamine/azide, O-alkylated hydroxylamine/alkyne, alkylated hydrazine/azide, alkylated hydrazine/alkyne, acylated hydrazine/azide, acylated hydrazine/alkyne, azide/aniline, alkyne/aniline,

or a combination of ketone with one of the groups of

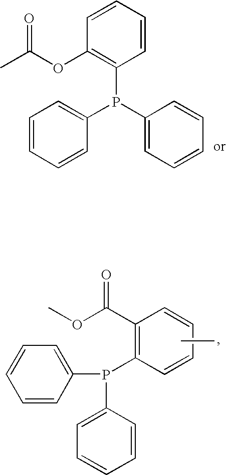

a combination of aldehyde with one of the groups of

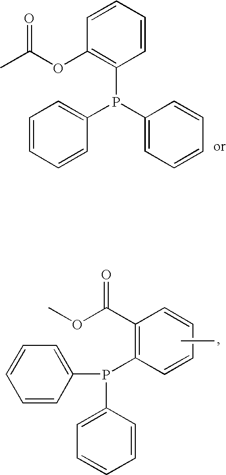

a combination of 1,2-diol with one of the groups of

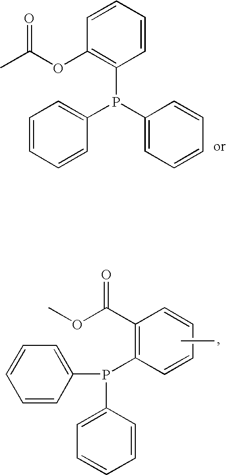

a combination of 1,2-aminoalcohol with one of the groups of

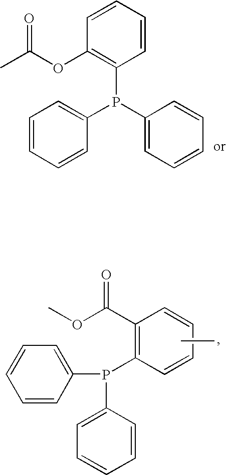

or a combination of aniline with one of the groups of

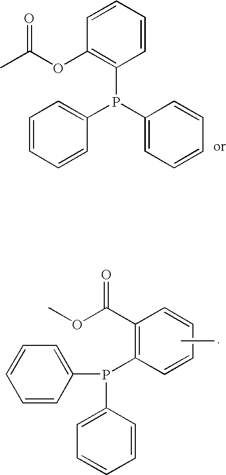

### Embodiment 5

A method according to any of embodiments 1 to 4, wherein the building block comprises a combination of two attachment chemical groups selected from the list of: ketone/azide or ketone/alkyne.

### Embodiment 6

A method according to any of embodiments 1 to 5, wherein the incorporation chemical group is an amine.

### Embodiment 7

A method of embodiment 6 wherein the incorporation chemical group is an amine and the attachment chemical groups comprise a combination of two chemical groups selected from ketone/1,2-diol, ketone/1,2-aminoalcohol, ketone/azide, ketone/alkyne, aldehyde/1,2-diol, aldehyde/1,2-aminoalcohol, aldehyde/azide, aldehyde/alkyne, 1,2-diol/azide, 1,2-diol/alkyne, 1,2-diol/aniline, 1,2-aminoalcohol/azide, 1,2-aminoalcohol/alkyne, 1,2-aminoalcohol/aniline, O-alkylated hydroxylamine/azide, O-alkylated hydroxylamine/alkyne, alkylated hydrazine/azide, alkylated hydrazine/alkyne, acylated hydrazine/azide, acylated hydrazine/alkyne, azide/aniline, alkyne/aniline,

a combination of ketone with a chemical group comprising

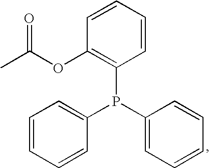

a combination of ketone with a chemical group comprising

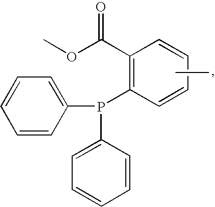

a combination of aldehyde with a chemical group comprising

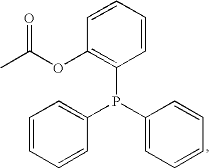

a combination of aldehyde with a chemical group comprising

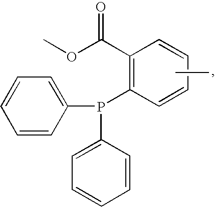

a combination of 1,2-diol with a chemical group comprising

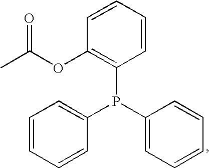

a combination of 1,2-diol with a chemical group comprising

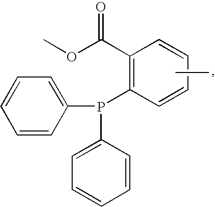

a combination of 1,2-aminoalcohol with a chemical group comprising

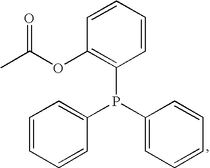

a combination of 1,2-aminoalcohol with a chemical group comprising

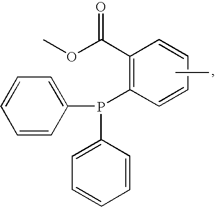

a combination of aniline with a chemical group comprising

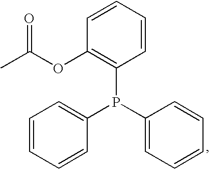

or a combination of aniline with a chemical group comprising

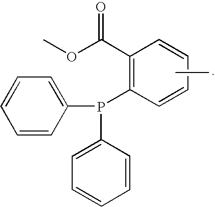

### Embodiment 8

A method according to any of embodiments 1 to 5, wherein the incorporation chemical group is an alpha amino amide.

### Embodiment 9

A method according to embodiment 8, wherein the incorporation chemical group is an alpha-amino amide and the attachment chemical groups comprise a combination of ketone/azide or ketone/alkyne.

### Embodiment 10

A method of any of embodiments 1 to 9, wherein the building block has the structural formula of

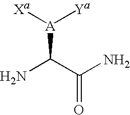

wherein A is any triradical moiety and the combination of X^{a} and Y^{a} are the two attachment chemical groups.

### Embodiment 11

A method according to embodiment 10, wherein the formula for the triradical moiety A comprises the formula

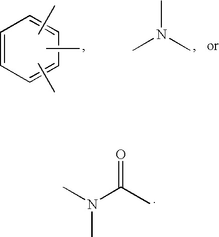

### Embodiment 11

A method according to embodiment 10, wherein the building block has the structural formula of

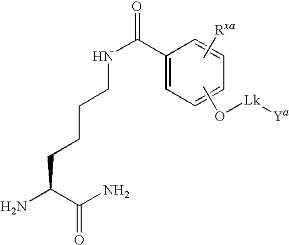

wherein Lk is a biradical of an alkane, alkene, arene, or heteroarene group, and R^{xa} is a chemical group comprising the attachment group X^{a}.

### Embodiment 12

A method according to embodiment 11, wherein Lk is a biradical of an alkane.

### Embodiment 13

A method according to embodiment 10, wherein the building block has the structural formula of

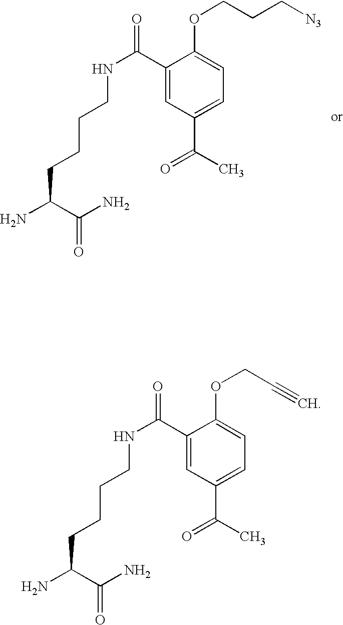

### Embodiment 14

A method according to any of embodiments 1 to 13, wherein the C-terminal amino acid of said peptide to be derivatised is selected from the group consisting of isoleucine, phenylalanine, leucine, tryptophan, alanine methionine, valine, and glycine.

### Embodiment 15

A method according to embodiment 14, wherein the C-terminal amino acid of said peptide is an alanine.

### Embodiment 16

A method according to any of embodiments 1 to 15, wherein the enzyme is carboxypeptidase Y or a variant thereof, which variant retains the ability to catalyse a reaction, by which the C-terminal amino acid of a protein or peptide is replaced by a different chemical moiety, or a fragment thereof, which fragment retains the ability to catalyse a reaction, by which the C-terminal amino acid of a protein or peptide is replaced by a different chemical moiety.

### Embodiment 17

A method according to embodiment 16, wherein the enzyme is carboxypeptidase Y.

### Embodiment 18

A method according to any one of embodiments 1 to 17, wherein the peptide is a peptide useful in therapy.

### Embodiment 19

A method according to any one of embodiments 1 to 18, wherein the peptide is human growth hormone.

### Embodiment 20

A method according to any one of embodiments 1 to 18, wherein the peptide is a derivative of human growth hormone.

### Embodiment 21

A method according to any one of embodiments 1 to 18, wherein the peptide is a variant of human growth hormone or a derivative of a variant of human growth hormone.

### Embodiment 22

A method according to any one of embodiments 1 to 18, wherein the peptide is hGH-Leu-Ala.

### Embodiment 23

A compound produced by a method according to any one of embodiments 1 to 22.

### Embodiment 24

A compound obtainable by use of a method according to any one of embodiments 1 to 22.

### Embodiment 25

A pharmaceutical composition comprising a compound produced by a method according to any one of embodiments 18 to 22 and a pharmaceutically acceptable carrier or excipient.

### Embodiment 26

A pharmaceutical composition comprising a compound obtainable by use of a method according to any one of embodiments 18 to 22 and a pharmaceutically acceptable carrier or excipient.

### Embodiment 27

A compound according to any of embodiments 18 to 22 for the treatment and/or diagnosis of a disorder or disease of a patient, which disease or disorder is dependent on the therapeutic use of said peptide.

### Embodiment 28

Use of a compound produced by a method according to any one of embodiments 18 to 22 for the manufacture of a pharmaceutical composition for the treatment and/or diagnosis of a disorder or disease of a patient, which disease or disorder is dependent on the therapeutic use of said peptide.

### Embodiment 29

Use of a compound obtainable by use of a method according to any one of embodiments 18 to 22 for the manufacture of a pharmaceutical composition for the treatment and/or diagnosis of a disorder or disease of a patient, which disease or disorder is dependent on the therapeutic use of said peptide.

### Embodiment 30

A method for the production of a pharmaceutical composition useful for treatment and/or diagnosis of a disorder or disease of a patient, which method comprises
a) obtaining a peptide useful in the treatment and/or diagnosis of said disease or disorder,
b) derivatising said peptide according to a method according to any of embodiments 1 to 22, and
c) formulating said derivatised peptide into a pharmaceutically acceptable composition.

### Embodiment 31

A pharmaceutical composition prepared by use of a method according to embodiment 30.

### Embodiment 32

A compound having the structural formula of

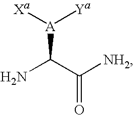

wherein A is any triradical moiety, and
X^{a} and Y^{a} are chosen such that the combination of X^{a} and Y^{a}, or Y^{a} and X^{a}, are selected from a combination of ketone/1,2-diol, ketone/1,2-aminoalcohol, ketone/azide, ketone/alkyne, aldehyde/1,2-diol, aldehyde/1,2-aminoalcohol, aldehyde/azide, aldehyde/alkyne, 1,2-diol/azide, 1,2-diol/alkyne, 1,2-diol/aniline, 1,2-aminoalcohol/azide, 1,2-aminoalcohol/alkyne, 1,2-aminoalcohol/aniline, O-alkylated hydroxylamine/azide, O-alkylated hydroxylamine/alkyne, alkylated hydrazine/azide, alkylated hydrazine/alkyne, acylated hydrazine/azide, acylated hydrazine/alkyne, azide/aniline, alkyne/aniline,
a combination of ketone with a chemical group comprising

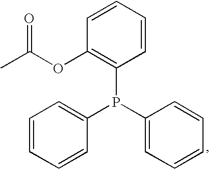

a combination of ketone with a chemical group comprising

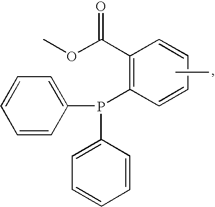

a combination of aldehyde with a chemical group comprising

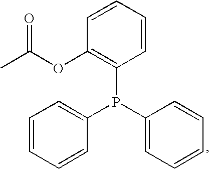

a combination of aldehyde with a chemical group comprising

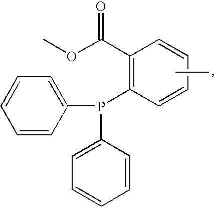

a combination of 1,2-diol with a chemical group comprising

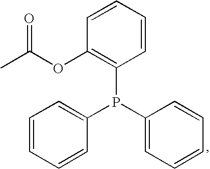

a combination of 1,2-diol with a chemical group comprising

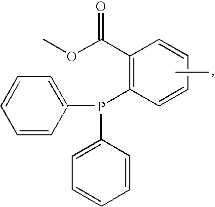

a combination of 1,2-aminoalcohol with a chemical group comprising

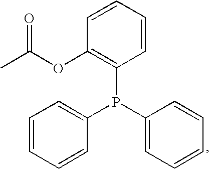

a combination of 1,2-aminoalcohol with a chemical group comprising

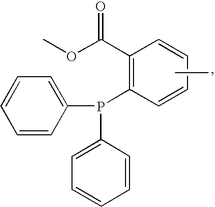

a combination of aniline with a chemical group comprising

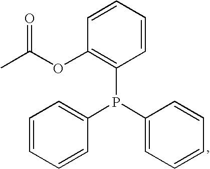

or a combination of aniline with a chemical group comprising

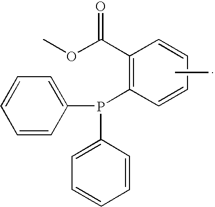

### Embodiment 33

A compound according to embodiment 32 having the structural formula of

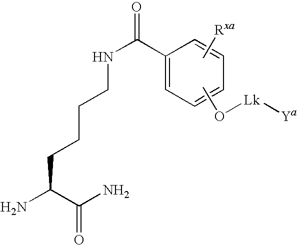

wherein Lk is a biradical of an alkane, alkene, arene, or heteroarene group, and
R^{xa} is a chemical group comprising the attachment group X^{a}, wherein X^{a} and Y^{a} are as defined in embodiment 32.

### Embodiment 34

A compound according to embodiment 33, wherein R^{xa} is X^{a}.

### Embodiment 34

A compound according to embodiment 33 or embodiment 34, wherein Lk is a biradical of alkane, and the combination of X^{a} and Y^{a}, or Y^{a} and X^{a}, are selected from a combination of ketone/azide, ketone/alkyne, aldehyde/azide, or aldehyde/alkyne.

### Embodiment 36

A compound having the structural formula

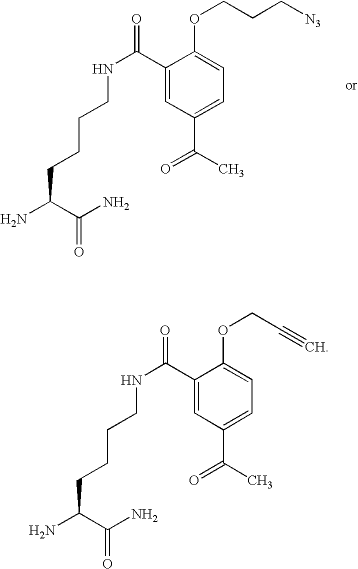

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims and/or aspects appended hereto as permitted by applicable law.

The present invention is further illustrated by the following examples which, however, are not to be construed as limiting the scope of protection. The features disclosed in the foregoing description and in the following examples may, both separately or in any combination thereof, be material for realising the invention in diverse forms thereof.

### EXAMPLES

### HPLC Method 02-b4-4

The RP-analyses was performed using an Alliance Waters 2695 system fitted with a Waters 2487 dualband detector. UV detections at 214 nm and 254 nm were collected using a Symmetry 300 C18, 5 um, 3.9 mm×150 mm column, 42° C. The compounds are eluted with a linear gradient of 5-95% acetonitrile in water which is buffered with 0.05% trifluoroacetic acid over 15 minutes at a flow-rate of 1.0 min/min.

HPLC Method 03-b6-1:

HPLC (Method 03_B6_{—}1): The RP-analysis was performed using a Waters 2690 systems fitted with a Waters 996 diode array detector. UV detections were collected at 214, 254, 276, and 301 nm on a 218TP54 4.6 mm×250 mm 5μ C-18 silica column (The Seperations Group, Hesperia), which was eluted at 0.5 ml/min at 42° C. The column was equilibrated with 5% acetonitrile (+0.1% TFA) in an aqueous solution of TFA in water (0.1%). After injection, the sample was eluted by a gradient of 0% to 90% acetonitrile (+0.1% TFA) in an aqueous solution of TFA in water (0.1%) during 50 min.

Mass spectra for peptides were obtained on an Agilent 1100 Series in the range of 500-1800 Da or on Perkin Elmer PE API 100 in the range of 500-2000 Da. Typically the found signals for m/z correspond to a series of any of z=1, 2, 3, 4, 5, or 6.

MALDI-TOF spectra were obtained on a Bruker Daltonix autoflex.

### Example 1

### (S)-6-(2-(3-(4-(3-((2-(2-(2-(2-(2-(2-(2-(2-(Acetylamino)ethoxy)ethoxy)ethoxy)ethoxy)ethoxy)-ethoxy)ethoxy)ethyl)carbamoyl)phenoxymethyl)triazol-1-yl)propoxy)-5-(1-(4-(4-((4-methyl-2-oxo-2H-chromen-7-yloxy)acetylamino)butoximino)ethyl)benzoylamino)-2-((((((((glutam-1-yl)aspart-1-yl)asparaginyl)glutam-1-yl)phenylalanyl)phenylalanyl)leucyl)amino)hexanoic amide

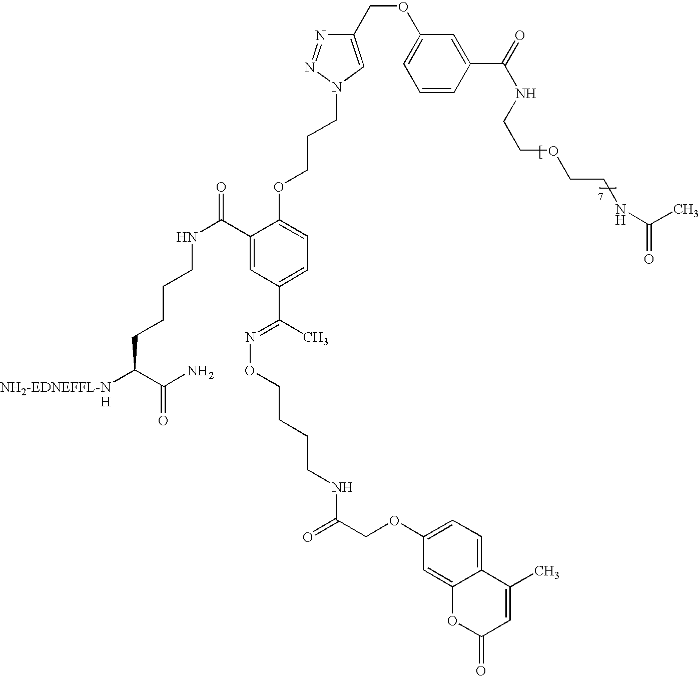

### Step 1

### (S)-6-(5-acetyl-2-(3-azidopropoxy)benzoylamino)-2-((((((((glutam-1-yl)aspart-1-yl)-asparaginyl)glutam-1-yl)phenylalanyl)phenylalanyl)leucyl)amino)hexanoic amide

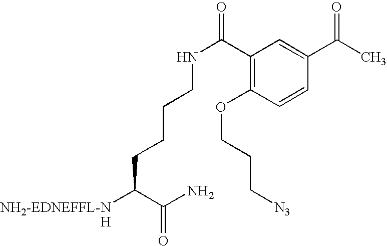

A solution consisting of (((((((glutam-1-yl)aspart-1-yl)asparaginyl)glutam-1-yl)-phenylalanyl)phenylalanyl)leucyl)alanine (16 mg, 0.016 mmol), which was synthesized as described in step 1 of example 4, 5-acetyl-N-((S)-5-amino-5-carbamoylpentyl)-2-(3-azidopropoxy)benzamide (1.270 g, 3.25 mmol)), which was prepared as described in example 5, and beta-hydroxypropylcyclodextrin (320 mg) in a buffer (10.5 ml) consisting of 250 mM HEPES and 5 mM EDTA, which had been adjusted to pH 7.5 by addition of hydrochloric acid. The pH was adjusted to 7.98 by addition of 1 N sodium hydroxide (2.50 ml). Buffer (2.93 ml), consisting of 250 mM HEPES and 5 mM EDTA, which had been adjusted to pH 7.5 by addition of hydrochloric acid, was added, obtaining a total volume of 15.93 ml with a pH of 7.92. A solution of CPY in water (200 U/ml, 0.32 ml, 64 U) was added. The reaction mixture was shaken gently at room temperature for 17 h. It was diluted with water and immediately subjected to a reversed phase HPLC-chromatography on a C18-column using a gradient of 10-50% acetonitrile, in water, which was buffered with 0.1% trifluoroacetic acid, as eluent. The fractions, containing the desired product were combined, diluted with water and lyophilized to give 6.5 mg of (S)-6-(5-acetyl-2-(3-azidopropoxy)benzoylamino)-2-((((((((glutam-1-yl)aspart-1-yl)asparaginyl)glutam-1-yl)-phenylalanyl)phenylalanyl)leucyl)amino)hexanoic amide.

HPLC: Rₜ=7.46 min (method 02-b4-4).

MS: m/z=1285 (required for M+1: 1285).

### Step 2

### (2-(2-(2-(2-(2-(2-(2-(2-Acetylaminoethoxy)ethoxy)ethoxy)ethoxy)ethoxy)ethoxy)ethoxy)ethyl)-carbamic acid tert-butyl ester

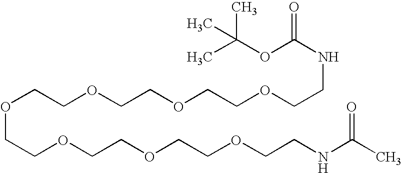

(2-(2-(2-(2-(2-(2-(2-(2-Aminoethoxy)ethoxy)ethoxy)ethoxy)ethoxy)ethoxy)ethoxy)-ethyl)carbamic acid tert-butyl ester (commercially available from e.g. Fluka, 1 g, 2.134 mmol) and triethylamine (0.297 ml, 2.134 mmol) were dissolved in dichloromethane (100 ml). Acetic anhydride (0.202 ml, 2.134 mmol) was added. The reaction mixture was stirred for 16 h at room temperature. It was diluted with ethyl acetate (300 ml) and washed with a 10% aqueous solution of sodium hydrogen sulphate (150 ml). The aqueous phase was extracted with ethyl acetate (2×100 ml). The combined organic layers were washed with brine (200 ml) and dried over sodium sulphate. The solvent was removed in vacuo to give 790 mg of crude (2-(2-(2-(2-(2-(2-(2-(2-acetylaminoethoxy)ethoxy)ethoxy)ethoxy)ethoxy)-ethoxy)ethoxy)ethyl)carbamic acid tert-butyl ester, which was used without further purification.

MS: M/Z=511 (required for M+1: 511); 533 (required for M+Na: 533), 411 (required for M-Boc: 411).

¹H-NMR (CDCl₃): δ=1.45 (s, 9H); 1.95 (s, 3H); 3.30 (m, 2H); 3.45 (m, 2H); 3.50-3.80 (m, 28H); 5.10 (br, 1H); 6.35 (br, 1H).

### Step 3

### N-(2-(2-(2-(2-(2-(2-(2-(2-Aminoethoxy)ethoxy)ethoxy)ethoxy)ethoxy)ethoxy)ethoxy)ethyl)-acetamide

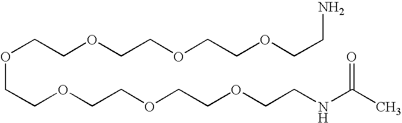

Crude (2-(2-(2-(2-(2-(2-(2-(2-acetylaminoethoxy)ethoxy)ethoxy)ethoxy)ethoxy)-ethoxy)ethoxy)ethyl)carbamic acid tert-butyl ester (790 mg, 1.54 mmol) was dissolved in dichloromethane (25 ml). Trifluoroacetic acid (25 ml) was added. The reaction mixture was stirred at room temperature for 1.5 h. The solvent was removed in vacuo. The residue was dissolved in dichloromethane (50 ml). The solvent was removed in vacuo. The latter procedure was repeated twice to give 970 mg of the trifluoroacetic acid salt of N-(2-(2-(2-(2-(2-(2-(2-(2-aminoethoxy)ethoxy)ethoxy)ethoxy)ethoxy)ethoxy)ethoxy)ethyl)acetamide.

HPLC: Rₜ=3.62 min (method 02-b4-4).

MS: m/z=411 (required for M+1: 411).

¹H-NMR (CDCl₃): δ=2.11 (s, 3H); 3.15 (m, 2H); 3.45-3.80 (m, 30H); 7.0 (br, 3H); 7.77 (br, 1H).

### Step 4

### N-(2-(2-(2-(2-(2-(2-(2-(2-Acetylaminoethoxy)ethoxy)ethoxy)ethoxy)ethoxy)ethoxy)ethoxy)-ethyl)-3-(prop-2-ynyloxy)benzamide

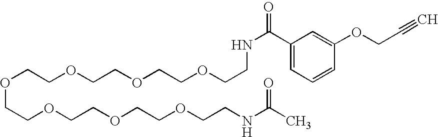

3-Prop-2-ynyloxy-benzoic acid 2,5-dioxopyrrolidin-1-yl ester (256 mg, 0.94 mmol) was added to a solution of N-(2-(2-(2-(2-(2-(2-(2-(2-aminoethoxy)ethoxy)ethoxy)ethoxy)-ethoxy)ethoxy)ethoxy)ethyl)acetamide (320 mg, 0.78 mmol) in dichloromethane (20 ml). Triethylamine (2.67 ml, 15.6 mmol) was added. The reaction mixture was stirred for 23 h at room temperature. It was extracted with a saturated aqueous solution of sodium hydrogencarbonate (20 ml). The aqueous phase was extracted with dichloromethane (2×30 ml). The combined organic layers were dried over sodium sulphate. The solvent was removed in vacuo The residue was dissolved in acetonitrile (4 ml) Water (10 ml) was added and the formed precipitation was removed by filtration. The solvent was removed in vacuo. The residue was dissolved in water (15 ml) and subjected to a reversed phase HPLC-chromatography on a C18-column, using a gradient of 5-30% acetonitrile in water, which was buffered with 0.1% trifluoroacetic acid. The fractions, containing the desired compound were pooled and lyophilized to give 70 mg of N-(2-(2-(2-(2-(2-(2-(2-(2-acetylaminoethoxy)ethoxy)-ethoxy)ethoxy)ethoxy)ethoxy)ethoxy)ethyl)-3-(prop-2-ynyloxy)benzamide.

HPLC: Rₜ=6.49 min (method 02-b4-4).

MS: m/z=569 (required for M+1: 569); 592 (required for M+Na: 592).

¹H-NMR (DMSO-d₆): δ=1.79 (s, 3H); 3.00-3.60 (m, 33H); 4.84 (s, 2H); 7.12 (m, 1H); 7.40 (m, 3H); 7.85 (br, 1H); 8.47 (br, 1H).

### Step 5

### (S)-6-(2-(3-(4-(3-((2-(2-(2-(2-(2-(2-(2-(2-(Acetylamino)ethoxy)ethoxy)ethoxy)ethoxy)ethoxy)-ethoxy)ethoxy)ethyl)carbamoyl)phenoxymethyl)triazol-1-yl)propoxy)-5-acetylbenzoylamino)-2-((((((((glutam-1-yl)aspart-1-yl)asparaginyl)glutam-1-yl)phenylalanyl)phenylalanyl)leucyl)-amino)hexanoic amide

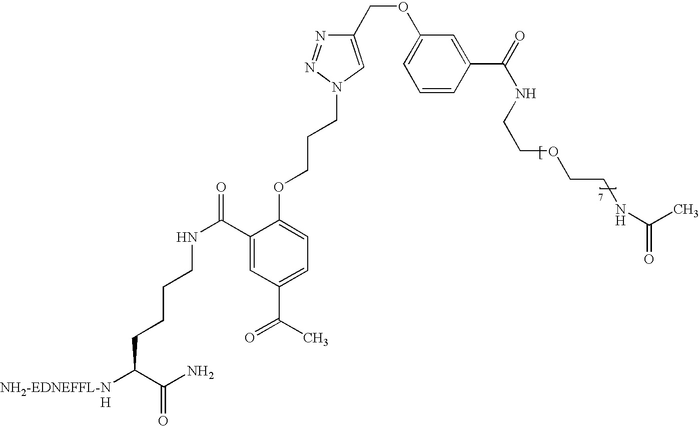

A copper(I)-salt solution was prepared by addition of a solution of copper(II) sulphate pentahydrate (20 mg, 0.080 mmol) in water (2 ml) to a solution of L-(+)-ascorbic acid (73 mg, 0.414 mmol) in a buffer consisting of 2% 2,4,6-lutidine in water (2 ml). This mixture was shaken and left for 1 min. 2.33 ml of this solution was taken and was added to a solution, which had been prepared by addition of a solution of N-(2-(2-(2-(2-(2-(2-(2-(2-acetylaminoethoxy)ethoxy)ethoxy)ethoxy)ethoxy)ethoxy)ethoxy)ethyl)-3-(prop-2-ynyloxy)-benzamide (25.5 mg, 47000 nmol) in a buffer (1 ml), consisting of 2% 2,4,6-lutidine in water, to a solution of (S)-6-(5-Acetyl-2-(3-azidopropoxy)benzoylamino)-2-((((((((glutam-1-yl)aspart-1-yl)asparaginyl)glutam-1-yl)phenylalanyl)phenylalanyl)leucyl)amino)hexanoic amide (6 mg, 4667 nmol) in a buffer (2 ml), consisting of 2% 2,4,6-lutidine in water. The reaction mixture was left for 16 h at room temperature. It was diluted with water to a total volume of 20 ml and subjected to a reverse phase HPLC-chromatography on a C18-column, using a gradient of 10-50% acetonitrile in water, which both was buffered with 0.1% trifluoroacetic acid. The fractions, which contained the desired compound were pooled and diluted with water (2 ml). The lyophilized to give 4.3 mg of (S)-6-(2-(3-(4-(3-((2-(2-(2-(2-(2-(2-(2-(2-(acetylamino)-ethoxy)ethoxy)ethoxy)ethoxy)ethoxy)ethoxy)ethoxy)ethyl)carbamoyl)phenoxymethyl)triazol-1-yl)propoxy)-5-acetylbenzoylamino)-2-((((((((glutam-1-yl)aspart-1-yl)asparaginyl)glutam-1-yl)phenylalanyl)phenylalanyl)leucyl)amino)hexanoic amide.

HPLC: Rₜ=7.13 min (method 02-b4-4).

MS: m/z=1856 (required for M+1: 1854), 928 (required for M+2²⁺: 928).

### Step 6

### 2-(4-(tert-Butoxycarbonylaminoxy)butyl)isoindole-1,3-dione

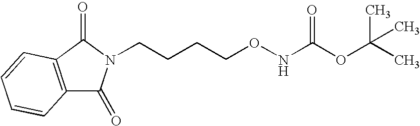

To a mixture of commercially available N-(4-bromobutyl)phthalimide (2.82 g, 10 mmol) and N-Boc-hydroxylamine (2.08 g, 15.6 mmol) was added acetonitrile (2 ml) and successively 1,8-diazabicyclo[5.4.0]undec-7-ene (2.25 ml, 15 mmol). The reaction mixture was stirred at room temperature for 30 min and then at 50° C. for 2 days. It was diluted with a mixture of water (30 ml) and 1 N hydrochloric acid (20 ml). It was extracted with ethyl acetate (2×100 ml). The organic phase was washed with brine (50 ml) and was dried over magnesium sulphate. The crude product was purified by chromatography on silica (60 g), using a gradient of heptane/ethyl acetate 1:0 to 0:1 as eluent to give 2.08 g of 2-(4-(tert-butoxycarbonylaminoxy)butyl)isoindole-1,3-dione.

¹H NMR (DMSO-d₆) δ 1.36 (s, 9H), 1.50 (m, 2H), 1.67 (m, 2H), 3.58 (t, 2H), 3.68 (t, 2H), 7.85 (m, 4H), 9.90 (s, 1H).

### Step 7

### N-(4-Aminobutoxy)carbamic acid tert-butyl ester

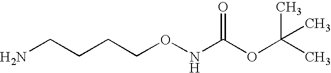

Hydrazine hydrate (1.0 ml, 20 mmol) was added to a solution of 2-(4-(tert-butoxy-carbonylaminoxy)butyl)isoindole-1,3-dione (2.08 g, 6.22 mmol) in ethanol (8.0 ml). The reaction mixture was stirred at 80° C. for 65 h. The solvent was removed in vacuo. The residue was dissolved in toluene (10 ml) and the solvent was removed in vacuo. The residue was suspended in 1 N hydrochloric acid (10 ml). The precipitation was removed by filtration and was washed with water (2 ml). The filtrate and the wash-liquids were combined and made basic with potassium carbonate. The solution was extracted with dichloromethane (4×20 ml). The organic layer was dried over magnesium sulphate. The solvent was removed in vacuo to give 0.39 g of N-(4-aminobutoxy)carbamic acid tert-butyl ester. Potassium carbonate (3 g) was added to the aqueous phase, which was extracted with dichloromethane (3×20 ml). These combined organic layers were dried over magnesium sulphate. The solvent was removed in vacuo to give another 0.39 g of N-(4-aminobutoxy)-carbamic acid tert-butyl ester.

¹H NMR (DMSO-d₆) δ 1.38 (m, 2H), 1.39 (s, 9H), 1.51 (m, 2H), 2.51 (t, 2H), 3.66 (t, 2H).

### Step 8

### (4-Methyl-2-oxo-2H-1-benzopyran-7-yloxy)acetic acid 2,5-dioxo-pyrrolidin-1-yl ester

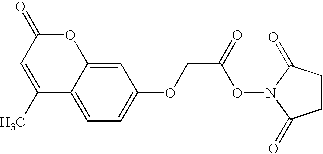

Commercially available (e.g. Aldrich) (4-methyl-2-oxo-2H-chromen-7-yloxy)acetic acid (1 g, 4.27 mmol) and triethylamine (0.595 ml, 4.27 mmol) were successively dissolved in N,N-dimethylformamide (25 ml). 2-Succinimido-1,1,3,3-tetramethyluronium tetrafluoroborate (1.29 g, 4.27 mmol) was added. The reaction mixture was stirred for 16 h at room temperature. It was diluted with ethyl acetate (250 ml) and washed with a 10% aqueous solution of sodium hydrogensulphate (200 ml). The aqueous phase was extracted with ethyl acetate (100 ml). The combined organic layers were washed with a mixture of brine (100 ml) and water (100 ml). They were dried over sodium sulphate. The solvent was removed in vacuo to give 1.38 g of crude (4-methyl-2-oxo-2H-1-benzopyran-7-yloxy)acetic acid 2,5-dioxo-pyrrolidin-1-yl ester.

MS: m/z=332 (required for M+1: 332), 354 (required for M+Na: 354).

¹H NMR (DMSO-d₆) δ 2.41 (s, 3H); 2.84 (s, 4H); 5.51 (s, 2H); 6.27 (s, 1H); 7.05 (m, 2H); 7.75 (d, 1H).

### Step 9

### (4-(2-(4-Methyl-2-oxo-2H-chromen-7-yloxy)acetylamino)butoxy)carbamic acid tert-butyl ester

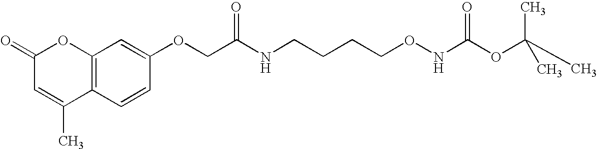

N-(4-Aminobutoxy)carbamic acid tert-butyl ester (1.30 g, 6.34 mmol) was added to a solution of crude (4-methyl-2-oxo-2H-1-benzopyran-7-yloxy)acetic acid 2,5-dioxo-pyrrolidin-1-yl ester (2.10 g, 6.34 mmol) in N,N-dimethylformamide (100 ml). Ethyldiiso-propylamine (6.51 ml, 38.0 mmol) was added. The reaction mixture was stirred for 20 h at room temperature. A 10% aqueous solution of sodium hydrogensulphate (200 ml) and ethyl acetate (300 ml) were added. The phases were separated. The aqueous phase was extracted with ethyl acetate (2×200 ml). The combined organic layers were washed with a saturated aqueous solution of sodium hydrogencarbonate (200 ml) and dried over sodium sulphate. The solvent was removed in vacuo to give crude (4-(2-(4-methyl-2-oxo-2H-chromen-7-yloxy)acetylamino)butoxy)carbamic acid tert-butyl ester.

HPLC: Rₜ=8.06 min (method 02-b4-4).

MS: m/z=321 (required for M-Boc: 321); 443 (required for M+Na: 443).

¹H NMR (CDCl₃) δ 1.47 (s, 9H); 1.69 (m, 4H); 2.42 (s, 3H); 3.41 (t, 2H); 3.89 (t, 2H); 4.55 (s, 2H); 6.19 (s, 1H); 6.86 (m, 2H); 7.15 (m, 2H); 7.56 (d, 1H).

### Step 10

### N-(4-Aminooxybutyl)-2-(4-methyl-2-oxo-2H-1-benzopyran-7-yloxy)acetamide

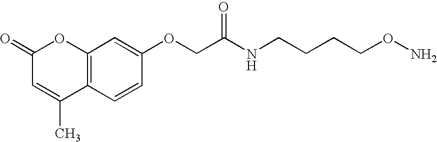

Crude (4-(2-(4-methyl-2-oxo-2H-chromen-7-yloxy)acetylamino)butoxy)carbamic acid tert-butyl ester (1.7 g, 4.04 mmol) was dissolved in dichloromethane (50 ml). Trifluoroacetic acid (50 ml) was added. The reaction mixture was stirred for 1.5 h at room temperature. The solvent was removed in vacuo. The residue was dissolved in dichloromethane (100 ml). The solvent was removed in vacuo. The latter procedure was repeated twice. The residue was suspended in water (15 ml). It was filtered and the filtrate was subjected to a reversed phase HPLC-chromatography on a C18-column using a gradient of 5-60% acetonitrile in water which was buffered with 0.1% trifluoroacetic acid as eluent. The fractions which contained the desired compound were pooled. The solvent was removed in vacuo. It was dissolved in water (15 ml) and lyophilized to give 440 mg of N-(4-aminooxybutyl)-2-(4-methyl-2-oxo-2H-1-benzopyran-7-yloxy)acetamide.

HPLC: Rₜ=4.76 min (method 02-b4-4).

MS: m/z=321 (required for M+1: 321); 346 (required for M+Na: 346).

¹H NMR (DMSO-d₆) δ 1.55 (m, 4H); 2.41 (s, 3H); 3.17 (t, 2H); 3.93 (t, 2H); 4.61 (s, 2H); 6.24 (s, 1H); 6.96 (m, 2H); 7.70 (d, 1H), 8.23 (s, 3H); 10-11 (br, 2H).

### Step 11

### (S)-6-(2-(3-(4-(3-((2-(2-(2-(2-(2-(2-(2-(2-(acetylamino)ethoxy)ethoxy)ethoxy)ethoxy)ethoxy)-ethoxy)ethoxy)ethyl)carbamoyl)phenoxymethyl)triazol-1-yl)propoxy)-5-(1-(4-((4-methyl-2-oxo-2H-chromen-7-yloxy)acetylamino)butoximino)ethyl)benzoylamino)-2-((((((((glutam-1-yl)aspart-1-yl)asparaginyl)glutam-1-yl)phenylalanyl)phenylalanyl)leucyl)amino)hexanoic amide

(S)-6-(2-(3-(4-(3-((2-(2-(2-(2-(2-(2-(2-(2-(Acetylamino)ethoxy)ethoxy)ethoxy)ethoxy)ethoxy)-ethoxy)ethoxy)ethyl)carbamoyl)phenoxymethyl)triazol-1-yl)propoxy)-5-acetylbenzoylamino)-2-((((((((glutam-1-yl)aspart-1-yl)asparaginyl)glutam-1-yl)phenylalanyl)phenylalanyl)leucyl)-amino)hexanoic amide (2 mg, 1078 nmol), which had been prepared in step 2, was dissolved in N,N-dimethylformamide (1 ml). Acetic acid (0.0015 ml, 27000 nmol) and N,N-dimethylformamide (0.5 ml) were added successively. A solution of N-(4-aminooxybutyl)-2-(4-methyl-2-oxo-2H-1-benzopyran-7-yloxy)acetamide (4.7 mg, 11000 nmol) in N,N-dimethylformamide (0.5 ml) was added. N,N-dimethylformamide (0.655 ml) was added. The reaction mixture was left at room temperature for 19 h. The HPLC showed a purity of 27% of the desired product. The reaction mixture was diluted with water to a total volume of 10 ml and subjected to a reversed phase HPLC-chromatography, using a gradient of 20-60% acetonitrile in water, which was buffered with 0.1% trifluoroacetic acid. The fractions, containing the desired compound were pooled and lyophilized to give (S)-6-(2-(3-(4-(3-((2-(2-(2-(2-(2-(2-(2-(2-(acetylamino)ethoxy)ethoxy)ethoxy)ethoxy)ethoxy)-ethoxy)ethoxy)ethyl)carbamoyl)phenoxymethyl)triazol-1-yl)propoxy)-5-(1-(4-((4-methyl-2-oxo-2H-chromen-7-yloxy)acetylamino)butoximino)ethyl)benzoylamino)-2-((((((((glutam-1-yl)aspart-1-yl)asparaginyl)glutam-1-yl)phenylalanyl)phenylalanyl)leucyl)amino)hexanoic amide.

HPLC: Rₜ=8.20 min (method 02-b4-4).

MS: m/z=1079.4 (required for M+2²⁺: 1079.0), 719.8 (required for M+3³⁺: 719.7)

### Example 2

### ((S)-5-Amino-1-(carbamoyl)pentyl)carbamic acid tert-butyl ester

### Step 1

### ((S)-5-(tert-Butoxycarbonylamino)-5-(carbamoyl)pentyl)carbamic acid benzyl ester

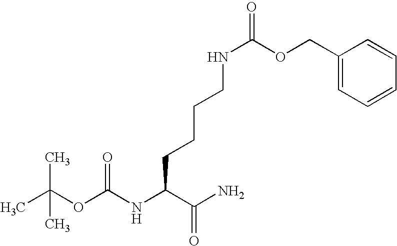

2,5-Dioxopyrrolidin-1-yl(S)-6-((benzyloxycarbonyl)amino)-2-((tert-butoxycarbonyl)-amino)hexanoate (commercially available at e.g. Fluka or Bachem, 15. g, 31 mmol) was dissolved in dichloromethane (50 ml). A 25% solution of ammonia in water was added. The reaction mixture was stirred vigorously for 16 h at room temperature. The solvent was removed in vacuo to yield 21.27 g of crude ((S)-5-(tert-butoxycarbonylamino)-5-(carbamoyl)-pentyl)carbamic acid benzyl ester, which was used in the next step without further purification.

¹H-NMR (DMSO-d₆): δ 1.2-1.6 (m, 6H); 1.37 (s, 9H); 2.95 (q, 2H); 3.80 (td, 1H); 5.00 (s, 2H); 6.70 (d, 1H); 6.90 (s, 1H); 7.20-7.40 (m, 7H).

MS: m/z=280.

### Step 2

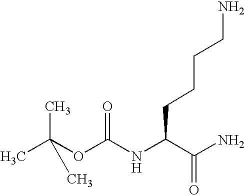

Crude ((S)-5-(tert-butoxycarbonylamino)-5-(carbamoyl)pentyl)carbamic acid benzyl ester (11.92 g, 31.41 mmol) was suspended in methanol (250 ml). Palladium on coal (50% wet) 1.67 g was added. The mixture was subjected to hydrogenation under pressure for 16 h. It was filtered through a plug of celite. The solvent was removed in vacuo to give 13.13 g of crude ((S)-5-amino-1-(carbamoyl)pentyl)carbamic acid tert-butyl ester, which was used in the next step without further purification.

¹H-NMR (DMSO-d₆): δ 1.30-1.60 (m, 6H); 1.37 (s, 9H); 2.65 (t, 2H); 3.80 (dt, 1H); 5.70 (br, 2H); 6.80 (d, 1H); 6.95 (s, 1H); 7.30 (s, 1H).

### Example 3

### (2S)-2-Amino-3-(4-(prop-2-ynyloxy)phenyl)propionamide

### Step 1

### (1-Carbamoyl-2-(4-hydroxyphenyl)ethyl)-carbamic acid tert-butyl ester

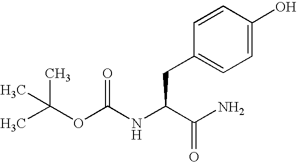

1-Hydroxybenzotriazole (5.44 g, 35.55 mmol) and 1-(3-dimethylaminopropyl)-3-ethylcarbodiimide hydrochloride (6.82 g, 35.55 mmol) were added successively to a solution of Boc-Tyr-OH (10 g, 35.55 mmol) in N,N-dimethylformamide (70 ml) and dichloromethane (70 ml). The reaction mixture was stirred for 20 min at room temperature. A 25% aqueous solution of ammonia (26.56 ml, 355 mmol) was added. The reaction mixture was stirred for 16 h at room temperature. It was diluted with ethyl acetate (300 ml) and washed with water (3×100 ml) and subsequently with a saturated aqueous solution of sodium hydrogen-carbonate (100 ml). The solvent was removed in vacuo to give 4.24 g of crude (1-Carbamoyl-2-(4-hydroxyphenyl)ethyl)carbamic acid tert-butyl ester.

¹H-NMR (DMSO-d₆): δ 1.31 (s 9H); 2.80 (dd, 1H); 2.83 (dd, 1H); 4.00 (m, 1H); 6.62 (d, 2H); 6.70 (d, 1H); 6.97 (br, 1H); 7.03 (d, 2H); 7.31 (br, 1H); 9.14 (s, 1H).

### Step 2

### ((S)-1-Carbamoyl-2-(4-(prop-2-ynyloxy)phenyl)ethyl)carbamic acid tert-butyl ester

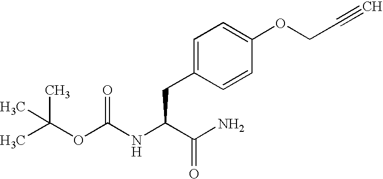

A mixture of ((S)-1-carbamoyl-2-(4-hydroxyphenyl)ethyl)-carbamic acid tert-butyl ester (1.0 g, 3.57 mmol), tetrabutylammonium iodide (65 mg, 0.17 mmol), potassium carbonate (3.94 g, 29 mmol), propargyl bromide (0.38 ml, 4.28 mmol) and N,N-dimethyl-formamide (15 ml) was heated to 60° C. for 16 h. It was cooled to room temperature, diluted with water (30 ml) and acidified with a 10% aqueous solution of sodium hydrogensulphate. The mixture was extracted with ethyl acetate (2×100 ml). The combined organic layers were washed with a saturated aqueous solution of sodium hydrogencarbonate (200 ml) and dried over magnesium sulphate. The solvent was removed in vacuo. The crude product was purified by flash chromatography on silica (100 g), using a mixture of dichloromethane/-methanol (10:1) as eluent, to give 998 mg of ((S)-1-carbamoyl-2-(4-(prop-2-ynyloxy)phenyl)-ethyl)carbamic acid tert-butyl ester.

MS: m/z=341 (M+Na)⁺.

¹H-NMR (DMSO-d₆) δ 1.31 (s, 9H); 2.50 (s, 1H); 2.67 (dd, 1H); 2.91 (dd, 1H); 4.03 (m, 1H); 4.74 (s, 2H); 6.77 (d, 1H); 6.86 (d, 2H); 6.99 (s, 1H), 7.17 (d, 2H); 7.35 (s, 1H).

### Step 3

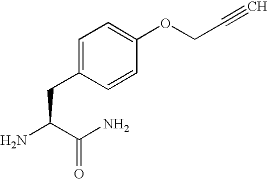

Trifluoroacetic acid (10 ml) was added to a solution of ((S)-1-carbamoyl-2-(4-(prop-2-ynyloxy)phenyl)ethyl)carbamic acid tert-butyl ester (998 mg, 3.13 mmol) in dichloromethane (10 ml). The reaction mixture was stirred for 1.5 h at room temperature. The solvent was removed. The residue was dissolved in dichloromethane (30 ml). The solvent was removed. The latter procedure was repeated twice to give 1.53 g of the trifluoroacetate salt of (2S)-2-amino-3-(4-(prop-2-ynyloxy)phenyl)propionamide.

MS: m/z=219 (M+1)⁺.

¹H-NMR (CDCl₃) δ 2.51 (s, 1H); 3.02 (m, 2H); 3.90 (m, 1H); 4.78 (s, 2H); 6.95 (d, 2H); 7.20 (d, 2H); 7.56 (s, 1H); 7.87 (s, 1H); 8.10 (br, 3H).

HPLC (method 02-B4-4): R_{f}=5.62 min.

### Example 4

### (S)-6-(5-Acetyl-2-(prop-2-ynyloxy)benzoylamino)-2-(((((((glutamyl)aspartyl)asparaginyl)-glutamyl)phenylalanyl)phenylalanyl)leucylamino)hexanoic amide

### Step 1

### ((((((Glutamyl)aspartyl)asparaginyl)glutamyl)phenylalanyl)phenylalanyl)leucyl)alanine

(((((((Glutamyl)aspartyl)asparaginyl)glutamyl)phenylalanyl)phenylalanyl)leucyl)-alanine was prepared on an Applied Biosystems 433A Peptide Synthesizer by standard Fmoc-strategy, known to a person skilled in the art, starting with a commercially available Fmoc-Ala-Wang resin. Following amino acid derivatives were used:

| coupling no. | amino acid derivative |
|---|---|
| 1 | Fmoc-Leu-OH |
| 2 | Fmoc-Phe-OH |
| 3 | Fmoc-Phe-OH |
| 4 | Fmoc-Glu(OtBu)-OH |
| 5 | Fmoc-Asn(Trt)-OH |
| 6 | Fmoc-Asp(OtBu)-OH |
| 7 | Fmoc-Glu(OtBu)-OH |

A mixture of trifluoroacetic acid (10.6 ml), water (0.265 ml) and triisopropylsilane (0.265 ml) was added to the resin. It was shaken for 1.5 h. The liquid was collected. The resin was washed with trifluoroacetic acid (1 ml). The liquids were combined. The solution was concentrated under a stream of nitrogen. Ether (40 ml) was added. The precipitation was isolated by centrifugation. The crude product was purified on a reversed phase C₁₈-column on a HPLC, using a gradient of 2-30% acetonitrile in water in a buffer of 0.1% trifluoroacetic acid, as eluent.

MS: m/z=984

HPLC: Rₜ=19.40 min (method 03-B6-1).

### Step 2

### 5-Acetyl-2-(prop-2-ynyloxy)benzoic acid methyl ester

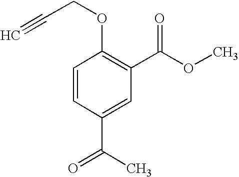

Methyl 5-acetylsalicylate (10 g, 51.5 mmol) was dissolved in N,N-dimethylformamide (100 ml). Potassium carbonate (21.4 g, 154 mmol) and tetrabutylammonium iodide (0.65 g, 2.58 mmol) were added successively. Propargylamine (4.88 ml, 56.6 mmol) was added. The reaction mixture was heated to 60° C. for 24 h. It was cooled to room temperature and diluted with water until all potassium carbonate was dissolved. Ethyl acetate (300 ml) was added. The phases were separated. The organic phase was washed with water (3×100 ml) and with a saturated aqueous solution of sodium hydrogencarbonate (100 ml). It was dried over sodium sulphate. The solvent was removed in vacuo. The product was recrystallized from ethyl acetate/heptane 2:3 to give 4.4 g of 5-acetyl-2-(prop-2-ynyloxy)-benzoic acid methyl ester.

MS: m/z=233 (required for M+1: 233), 255 (required for M+Na: 255).

¹H-NMR (CDCl₃) δ 2.57 (t, 1H); 2.60 (s, 3H); 3.93 (s, 3H); 4.88 (d, 2H); 7.20 (d, 1H); 8.13 (d, 1H); 8.43 (s, 1H).

### Step 3

### 5-Acetyl-2-(prop-2-ynyloxy)benzoic acid

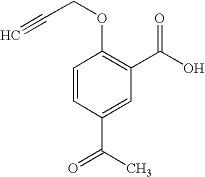

5-Acetyl-2-(prop-2-ynyloxy)benzoic acid methyl ester (2.9 g, 12.5 mmol) was dissolved in 1,4-dioxane (70 ml). A solution of lithium hydroxide (0.359 g, 15.0 mmol) in water (70 ml) was added. The reaction mixture was stirred at room temperature for 3 days. 1 N aqueous sodium hydroxide (30 ml) was added. The mixture was washed repeatedly with tert-butyl methyl ether (250 ml, 100 ml, 100 ml). The aqueous phase was acidified with a 10% aqueous solution of sodium hydrogensulphate to pH 2. It was repeatedly extracted with ethyl acetate (250 ml, 150 ml, 150 ml). The combined organic layers were dried over sodium sulphate. The solvent was removed in vacuo to give 2.64 g of 5-acetyl-2-(prop-2-ynyloxy)-benzoic acid.

MS: m/z=219 (required for M+1: 219), 241 (required for M+Na: 241).

### Step 4

### 5-Acetyl-2-(prop-2-ynyloxy)benzoic acid 2,5-dioxo-pyrrolidin-1-yl ester

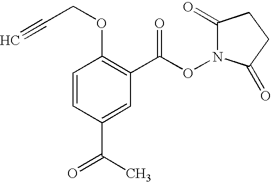

2-Succinimido-1,1,3,3-tetramethyluronium tetrafluoroborate (3.81 g, 12.1 mmol) was added to a solution of 5-acetyl-2-(prop-2-ynyloxy)benzoic acid (2.64 g, 12.1 mmol) and triethylamine (1.69 ml, 12.1 mmol) in N,N-dimethylformamide (200 ml). The reaction mixture was stirred for 21 h at room temperature. It was diluted with a 10% aqueous solution of sodium hydrogensulphate (200 ml) and extracted with ethyl acetate (200 ml). The aqueous phase was extracted with ethyl acetate (2×100 ml). The combined organic layers were dried over sodium sulphate. The solvent was removed in vacuo. The crude product was crystallised from ethyl acetate/heptane 1:2 to give 1.3 g of 5-acetyl-2-(prop-2-ynyloxy)benzoic acid 2,5-dioxo-pyrrolidin-1-yl ester.

MS: m/z=338 (required for M+Na: 338).

¹H-NMR (CDCl₃) δ 2.60 (t, 1H); 2.92 (m, 4H); 4.91 (d, 2H); 7.28 (d, 1H); 8.25 (d, 1H); 8.65 (s, 1H).

### Step 5

### ((S)-5-(5-Acetyl-2-(prop-2-ynyloxy)benzoylamino)-1-carbamoylpentyl)carbamic acid tert-butyl ester

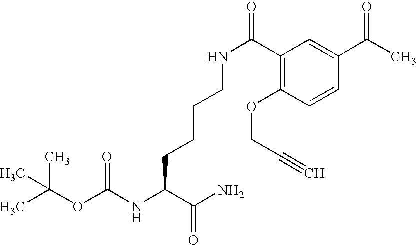

((S)-5-Amino-1-(carbamoyl)pentyl)carbamic acid tert-butyl ester (1.07 g, 4.34 mmol) was added to a solution of 5-acetyl-2-(prop-2-ynyloxy)benzoic acid 2,5-dioxo-pyrrolidin-1-yl ester (1.37 g, 4.34 mmol) in N,N-dimethylformamide (200 ml). Ethyldiisopropylamine (2.23 ml, 13.0 mmol) was added. The reaction mixture was stirred for 5 days at room temperature. It was diluted with ethyl acetate (400 ml) and washed with a 10% aqueous solution of sodium hydrogensulphate (250 ml). The aqueous phase was extracted with ethyl acetate (2×200 ml). The combined organic layers were washed with a saturated aqueous solution of sodium hydrogencarbonate (200 ml) and dried over sodium sulphate. The solvent was removed in vacuo to give 1.52 g of crude ((S)-5-(5-acetyl-2-(prop-2-ynyloxy)benzoylamino)-1-carbamoylpentyl)carbamic acid tert-butyl ester.

HPLC: Rₜ=7.13 min (method 02-b4-4).

MS: m/z=468 (required for M+Na: 468), 346 (required for M+1-Boc: 346).

¹H-NMR (DMSO-d₆) δ 1.30-1.60 (m, 6H); 1.37 (s, 9H); 2.55 (s, 3H); 3.25 (m, 2H); 3.69 (t, 1H); 3.85 (m, 1H); 5.02 (d, 2H); 6.75 (d, 1H); 6.93 (s, 1H); 7.23 (s, 1H); 7.30 (d, 1H); 7.95 (s, 1H); 8.05 (d, 1H); 8.20 (m, 1H).

### Step 6

### 5-Acetyl-N-((S)-5-amino-5-carbamoyl-pentyl)-2-(prop-2-ynyloxy)benzamide

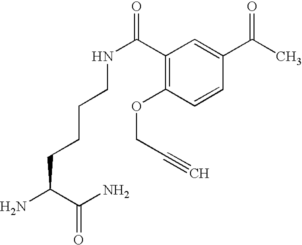

Crude ((S)-5-(5-acetyl-2-(prop-2-ynyloxy)benzoylamino)-1-carbamoylpentyl)-carbamic acid tert-butyl ester (1.52 g, 3.41 mmol) was dissolved in dichloromethane (30 ml). Trifluoroacetic acid (30 ml) was added. The reaction mixture was stirred for 1.75 h. The solvent was removed in vacuo. The residue was dissolved in dichloromethane (50 ml). The solvent was removed in vacuo. The latter procedure was repeated two times. The residue was dissolved in water and was subjected to a reversed phase HPLC-chromatography on a C18-column, using a gradient of 5-25 acetonitrile in water, which was buffered with 0.1% trifluoroacetic acid. The fractions containing the desired compound were pooled and lyophilized to give 670 mg of the trifluoroacetate salt of 5-acetyl-N-((S)-5-amino-5-carbamoyl-pentyl)-2-(prop-2-ynyloxy)benzamide.

HPLC: Rₜ=4.4 min (method: 0-2b4-4).

MS: m/z=346 (required for M+1: 346).

¹H-NMR (DMSO-d₆) δ 1.36 (m, 2H); 1.54 (m, 2H); 1.76 (m, 2H); 2.56 (s, 3H); 3.27 (m, 2H); 3.70 (m, 2H); 5.03 (s, 2H); 7.30 (d, 1H); 7.57 (br, 1H); 7.85 (br, 1H); 8.06 (br, 3H); 8.22 (m, 2H).

### Step 7

A solution of a trifluoroacetate salt of 5-acetyl-N-((S)-5-amino-5-carbamoyl-pentyl)-2-(prop-2-ynyloxy)benzamide (88 mg, 0.254 mmol) in a buffer (0.300 ml) consisting of 250 mM HEPES and 5 mM EDTA, which had been adjusted to pH 7.5 by addition of 1 N hydrochloric acid, was added to a solution of (((((((glutamyl)aspartyl)asparaginyl)glutamyl)-phenylalanyl)phenylalanyl)leucyl)alanine (1 mg, 1016 nmol) in a buffer (0.300 ml) consisting of 250 mM HEPES and 5 mM EDTA, which had been adjusted to pH 7.5 by addition of 1 N hydrochloric acid. Betahydroxypropylcyclodextrin (20 mg) was added to the solution. The solution was adjusted to pH 7.35 with 1 N sodium hydroxide (0.070 ml). It was adjusted to pH 6.70 by addition of concentrated hydrochloric acid (0.01 ml). A buffer (0.305 ml) consisting of 250 mM HEPES and 5 mM EDTA, which had been adjusted to pH 7.5 by addition of 1 N hydrochloric acid to obtain a total volume of 0.95 ml. A solution of CPY (200 U/ml, 0.020 ml, 4 U) was added to the mixture. After 45 min the reaction mixture was diluted with water and subjected to a reverse phase HPLC-chromatography on a C18-column, using a gradient of acetonitrile in water, which was buffered with 0.1% trifluoroacetic acid. (S)-6-(5-Acetyl-2-(prop-2-ynyloxy)benzoylamino)-2-(((((((glutamyl)aspartyl)asparaginyl)glutamyl)-phenylalanyl)phenylalanyl)leucylamino)hexanoic amid was identified.

MS: m/z=1240 (required for M+1: 1241).

### Example 5

### 5-Acetyl-N-((S)-5-amino-5-carbamoyl pentyl)-2-(3-azidopropoxy)benzamide

### Step 1

### 5-Acetyl-2-(3-bromo-propoxy)benzoic acid methyl ester

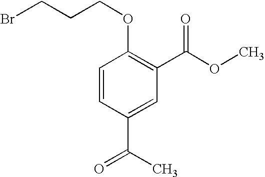

A mixture of methyl 5-acetylsalicylate (20 g, 103 mmol), potassium carbonate (42.7 g, 309 mmol), tetrabutylammonium iodide (1.90 g, 5.15 mmol), and N,N-dimethylformamide (150 ml) was added to a solution of 1,3-dibromopropane (42 ml, 412 mmol). The reaction mixture was heated to 60° C. for 16 h. It was cooled to room temperature. Water was added until all potassium carbonate was dissolved. It was extracted with ethyl acetate (300 ml). The organic layer was washed with water (3×200 ml) and a saturated aqueous solution of sodium hydrogencarbonate (200 ml). It was dried over sodium sulphate. The solvent was removed in vacuo. The crude product was purified by flash chromatography on silica (400 g), using a mixture of ethyl acetate/heptane/dichloromethane (1:2:1) as eluent to give 10.77 g of 5-acetyl-2-(3-bromo-propoxy)benzoic acid methyl ester.

MS: m/z=315 (M+1)

¹H-NMR (CDCl₃): δ 2.38 (quintet, 2H); 2.59 (s, 3H); 3.71 (t, 2H); 3.91 (s, 3H); 4.27 (t, 2H); 7.05 (d, 1H); 8.09 (d, 1H); 8.42 (s, 1H).

### Step 2

### 5-Acetyl-2-(3-azidopropoxy)benzoic acid methyl ester

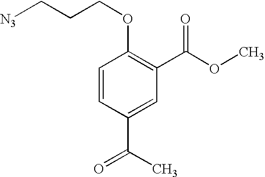

5-Acetyl-2-(3-bromo-propoxy)benzoic acid methyl ester (10.77 g, 34.2 mmol) was dissolved in N,N-dimethylformamide (200 ml). Sodium azide (4.44 g, 68.3 mmol) and tetrabutylammonium iodide (0.126 g, 0.342 mmol) were added successively. The reaction mixture was stirred at 60° C. for 16 h. It was cooled to room temperature and diluted with water (200 ml). It was extracted with ethyl acetate (300 ml). The organic layer was washed with water (3×200 ml) and dried over sodium sulphate. The solvent was removed in vacuo to yield the crude product. The aqueous phase was treated according to the literature (Lunn, G. Sansone E. B. Destruction of hazardous chemicals in the laboratory, 2^{nd} edition, John Wiley & Sons, New York) in order to remove the excess azide. The crude product was purified by flash chromatography on silica (90 g), using a mixture of ethyl acetate/heptane/-dichloromethane (3:2:1) to dissolve the crude product and a mixture of ethyl acetate/heptane (3:2) as eluent to give 5.15 g of 5-acetyl-2-(3-azidopropoxy)benzoic acid methyl ester.

MS: m/z=278 (M+1), 300 (M+Na), 249 (M−28).

¹H-NMR (CDCl₃): δ 2.12 (quintett, 2H); 2.58 (s, 3H); 3.62 (t, 2H); 3.91 (s, 3H); 4.21 (t, 2H); 7.03 (d, 1H); 8.10 (d, 1H); 8.42 (s, 1H).

### Step 3

### 5-Acetyl-2-(3-azidopropoxy)benzoic acid

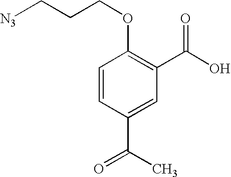

5-Acetyl-2-(3-azidopropoxy)benzoic acid methyl ester (5.15 g, 18.6 mmol) was dissolved in 1,4-dioxane (200 ml). A solution of lithium hydroxide (0.534 g, 22.3 mmol) in water (200 ml) was added. The reaction mixture was stirred for 4 days at room temperature. A 1 N aqueous solution of sodium hydroxide was added to obtain a solution with pH 13-14. It was washed with tert-butyl methyl ether (3×200 ml). The aqueous phase was acidified to pH 3 with a 10% aqueous solution of sodium hydrogensulphate. It was extracted with ethyl acetate (3×200 ml). The combined ethyl acetate layers were dried over sodium sulphate. The solvent was removed in vacuo to give crude 5-acetyl-2-(3-azidopropoxy)benzoic acid, which was used without further purification.

MS: m/z=236 (M−28), 286 (M+23).

¹H-NMR (CDCl₃): δ 2.19 (quintet, 2H); 2.63 (s, 3H); 3.64 (t, 2H); 4.37 (t, 2H); 7.12 (d, 1H); 8.22 (d, 1H); 8.70 (s, 1H); 10-12 (broad 1H).

HPLC: Rₜ=6.70 min (method 02-b4-4)

### Step 4

### 5-Acetyl-2-(3-azidopropoxy)benzoic acid 2,5-dioxo-pyrrolidin-1-yl ester

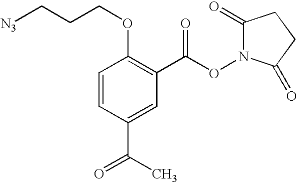

2-Succinimido-1,1,3,3-tetramethyluronium tetrafluoroborate (TSTU, 4.51 g, 15.0 mmol) was added to a solution of 5-acetyl-2-(3-azidopropoxy)benzoic acid (3.94, 15.0 mmol) and triethylamine (2.09 ml, 15.0 mmol) in N,N-dimethylformamide (100 ml). The reaction mixture was stirred for 24 h at room temperature. A 10% aqueous solution of sodium hydrogensulphate (400 ml) was added. The mixture was extracted with ethyl acetate (2×200 ml). The combined organic layers were washed with brine (200 ml) and dried over sodium sulphate. The solvent was removed in vacuo. It was treated with a mixture of ethyl acetate/heptane (2:1, 10 ml). The formed precipitation was isolated by filtration, washed with a mixture of ethyl acetate/heptane (2:1, 10 ml) and dried in vacuo to give 2.9 g of 5-acetyl-2-(3-azidopropoxy)benzoic acid 2,5-dioxo-pyrrolidin-1-yl ester.

MS: m/z=383 (M+Na), 333 (M+1-N₂).

¹H-NMR (CDCl₃): δ 2.12 (quintet, 2H); 2.61 (s, 3H); 2.92 (broad, 4H); 3.61 (t, 2H); 4.24 (t, 2H); 7.09 (d, 1H); 8.23 (d, 1H); 8.59 (s, 1H).

### Step 5

### ((S)-5-(5-Acetyl-2-(3-azidopropoxy)benzoylamino)-1-(carbamoyl)pentyl)carbamic acid tert-butyl ester

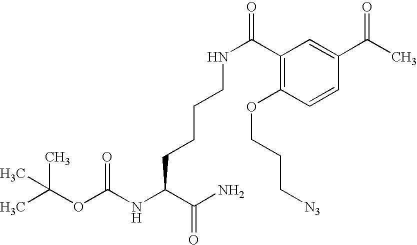

Crude (S)-5-amino-1-(carbamoyl)pentyl)carbamic acid tert-butyl ester (1.97 g, 8.05 mmol) was added to a solution of 5-acetyl-2-(3-azidopropoxy)benzoic acid 2,5-dioxo-pyrrolidin-1-yl ester (2.90 g, 8.05 mmol) and ethyldiisopropylamine (4.13 ml, 24.14 mmol) in N,N-dimethylformamide (200 ml). The reaction mixture was stirred for 16 h at room temperature. It was diluted with a 10% aqueous solution of sodium hydrogensulphate (400 ml) and extracted with ethyl acetate (2×300 ml). The combined organic layers were washed with a saturated aqueous solution of sodium hydrogencarbonate (300 ml) and dried over sodium sulphate. The solvent was removed in vacuo to give 3.01 g of crude ((S)-5-(5-acetyl-2-(3-azidopropoxy)benzoylamino)-1-(carbamoyl)pentyl)carbamic acid tert-butyl ester, which was used in the next step without further purification.

MS: m/z=391 (M+1-Boc).

¹H-NMR (DMSO-d₆): δ 1.29-1.65 (m, 6H); 1.36 (s, 9H); 2.05 (quintet, 2H); 2.55 (s, 3H); 3.26 (q, 2H); 3.55 (t, 2H); 3.84 (q, 1H); 4.23 (t, 2H); 6.70 (d, 1H); 6.93 (s, 1H); 7.24 (m, 2H); 8.02 (d, 1H); 8.10 (t, 1H); 8.18 (s, 1H).

HPLC: Rₜ=7.83 min (method 02-b4-4).

### Step 6

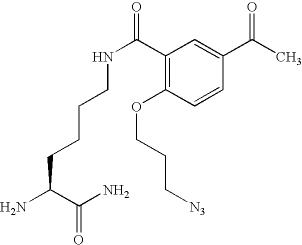

Trifluoroacetic acid (100 ml) was added to a mixture of ((S)-5-(5-acetyl-2-(3-azido-propoxy)benzoylamino)-1-(carbamoyl)pentyl)carbamic acid tert-butyl ester (3.01 g, 6 mmol) and dichloromethane (100 ml). The reaction mixture was stirred for 1.5 h at room temperature. The solvent was removed in vacuo. The residue was dissolved in dichloro-methane (100 ml). The solvent was removed in vacuo. This latter procedure was repeated two times. The crude product was purified on a HPLC reversed phase C₁₈-column, using a gradient of 0-40% acetonitrile in water, which was buffered with 0.1% trifluoroacetic acid, as eluent to give 1.40 g of the trifluoroacetate salt of 5-acetyl-N-((S)-5-amino-5-carbamoyl-pentyl)-2-(3-azidopropoxy)benzamide.

MS: m/z=391 (M+1).

¹H-NMR (DMSO-d₆): δ 1.36 (m, 2H); 1.52 (m, 2H); 1.73 (m, 2H); 2.05 (quintet, 2H); 2.55 (s, 3H); 3.27 (q, 2H); 3.55 (t, 2H); 3.69 (m, 1H); 4.24 (t, 2H); 7.25 (d, 1H); 7.57 (s, 1H); 7.83 (s, 1H); 8.03 (m, 4H); 8.18 (m, 2H).

HPLC: Rt=5.02 (method 02-b4-4).

1. A method of preparing a peptide, which peptide is capable of being derivatised with two property-modifying groups attached to the C-terminal of said peptide, comprising the step of
(a) bringing a building block, which is a chemical compound comprising two or more attachment chemical groups, which attachment chemical groups are not accessible in any of the amino acid residues constituting said peptide and which attachment chemical groups are different from each other, and a incorporation chemical group, which incorporation chemical group under certain circumstances is capable of reacting with the carboxyl group in the C-terminus of the peptide, into contact with the peptide in the presence of an enzyme capable of catalysing the incorporation of said building block into the C-terminus of said peptide by catalysing a reaction between the C-terminal carboxyl group and said incorporation chemical group.

2. A method of attaching two chemical moieties to the C-terminus of a peptide comprising the steps of
(a) bringing a building block, which is a chemical compound comprising two or more attachment chemical groups, which attachment chemical groups are not accessible in any of the amino acid residues constituting said peptide and which attachment chemical groups are different from each other, and a incorporation chemical group, which incorporation chemical group under certain circumstances is capable of reacting with the carboxyl group in the C-terminus of the peptide, into contact with the peptide in the presence of an enzyme capable of catalysing the incorporation of said building block into the C-terminus of said peptide by catalysing a reaction between the C-terminal carboxyl group and said incorporation chemical group,
(b) reacting in one or more steps a first attachment chemical group of the building block with a chemical group present on a first moiety to be attached to the peptide, which first moiety chemical group does not react with any functional groups present in the peptide and
(c) reacting in one or more steps a second attachment chemical group of the building block with a chemical group present on a second moiety to be attached to the peptide, which second moiety chemical group does not react with any functional groups present on the peptide.

3. The method according to claim 1, wherein the attachment groups of the building block are selected from the group consisting of ketones, aldehydes, 1,2-diols, 1,2-aminoalcohols, O-alkylated hydroxylamines, alkylated hydrazines, acylated hydrazines, azides, alkynes, anilines or comprise a group selected from 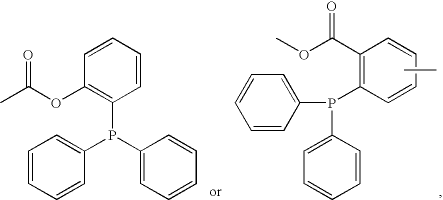
wherein the two attachment groups are different from each other.

4. The method according to claim 1, wherein the building block comprises a combination of two attachment chemical groups selected from ketone/1,2-diol, ketone/1,2-aminoalcohol, ketone/azide, ketone/alkyne, aldehyde/1,2-diol, aldehyde/1,2-aminoalcohol, aldehyde/azide, aldehyde/alkyne, 1,2-diol/azide, 1,2-diol/alkyne, 1,2-diol/aniline, 1,2-aminoalcohol/azide, 1,2-aminoalcohol/alkyne, 1,2-aminoalcohol/aniline, O-alkylated hydroxylamine/azide, O-alkylated hydroxylamine/alkyne, alkylated hydrazine/azide, alkylated hydrazine/alkyne, acylated hydrazine/azide, acylated hydrazine/alkyne, azide/aniline, alkyne/aniline,
or a combination of ketone with one of the groups of 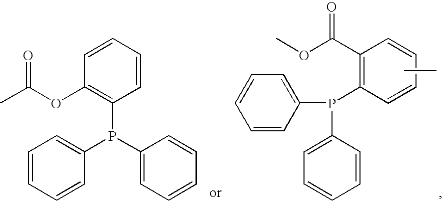
a combination of aldehyde with one of the groups of 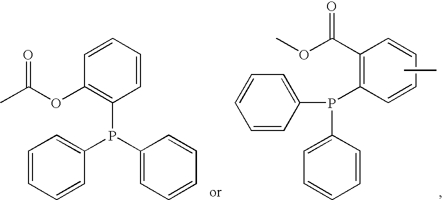
a combination of 1,2-diol with one of the groups of 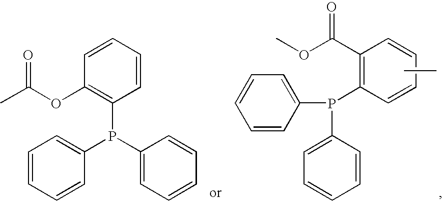
a combination of 1,2-aminoalcohol with one of the groups of 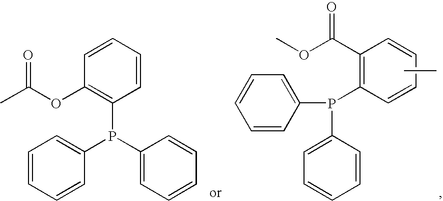
or a combination of aniline with one of the groups of

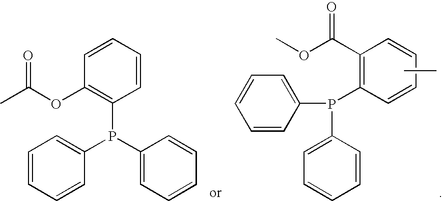

5. The method according to claim 1, wherein the incorporation chemical group is an amine.

6. The method of claim 5 wherein the incorporation chemical group is an amine and the attachment chemical groups comprise a combination of two chemical groups selected from ketone/1,2-diol, ketone/1,2-aminoalcohol, ketone/azide, ketone/alkyne, aldehyde/1,2-diol, aldehyde/1,2-aminoalcohol, aldehyde/azide, aldehyde/alkyne, 1,2-diol/azide, 1,2-diol/alkyne, 1,2-diol/aniline, 1,2-aminoalcohol/azide, 1,2-aminoalcohol/alkyne, 1,2-aminoalcohol/aniline, O-alkylated hydroxylamine/azide, O-alkylated hydroxylamine/alkyne, alkylated hydrazine/azide, alkylated hydrazine/alkyne, acylated hydrazine/azide, acylated hydrazine/alkyne, azide/aniline, alkyne/aniline,
a combination of ketone with a chemical group comprising 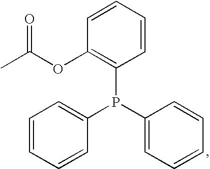
a combination of ketone with a chemical group comprising 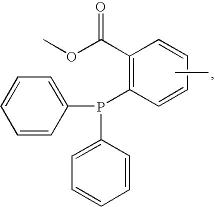
a combination of aldehyde with a chemical group comprising 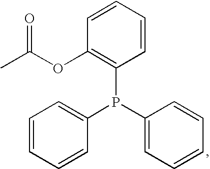
a combination of aldehyde with a chemical group comprising 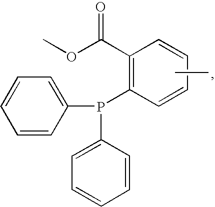
a combination of 1,2-diol with a chemical group comprising 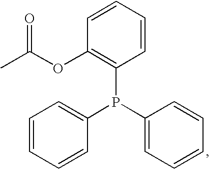
a combination of 1,2-diol with a chemical group comprising 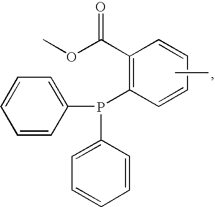
a combination of 1,2-aminoalcohol with a chemical group comprising 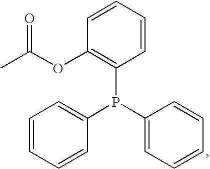
a combination of 1,2-aminoalcohol with a chemical group comprising 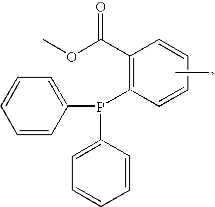
a combination of aniline with a chemical group comprising 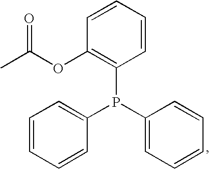
or a combination of aniline with a chemical group comprising

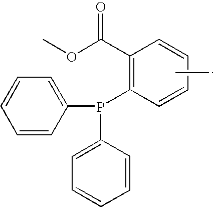

7. The method according to claim 1, wherein the building block has the structural formula of 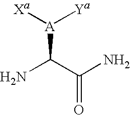
wherein A is any triradical moiety and the combination of X' and Y^{a} are the two attachment chemical groups.

8. The method according to claim 1, wherein the enzyme is carboxypeptidase Y or a variant thereof, which variant retains the ability to catalyse a reaction, by which the C-terminal amino acid of a protein or peptide is replaced by a different chemical moiety, or a fragment thereof, which fragment retains the ability to catalyse a reaction, by which the C-terminal amino acid of a protein or peptide is replaced by a different chemical moiety.

9. The method according to claim 1, wherein the peptide is a peptide useful in therapy.

10. The method according to claim 1, wherein the peptide is human growth hormone, a derivative of human growth hormone, or a variant of human growth hormone or a derivative of a variant of human growth hormone.

11. The method according to claim 1, wherein the peptide is hGH-Leu-Ala.

12. The A compound produced by a method according to claim 1.

13. (canceled)

14. A pharmaceutical composition comprising a compound produced by a method according to claim 1 and a pharmaceutically acceptable carrier or excipient.

15. (canceled)

16. (canceled)

17. (canceled)

18. A method for the production of a pharmaceutical composition useful for treatment and/or diagnosis of a disorder or disease of a patient, which method comprises
a) obtaining a peptide useful in the treatment and/or diagnosis of said disease or disorder,
b) derivatising said peptide according to a method according to claim 1, and
c) formulating said derivatised peptide into a pharmaceutically acceptable composition.

19. (canceled)

20. A compound having the structural formula of 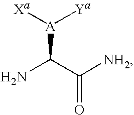
wherein A is any triradical moiety, and
X^{a} and Y^{a} are chosen such that the combination of X^{a} and Y^{a}, or Y^{a} and X^{a}, are selected from a combination of ketone/1,2-diol, ketone/1,2-aminoalcohol, ketone/azide, ketone/alkyne, aldehyde/1,2-diol, aldehyde/1,2-aminoalcohol, aldehyde/azide, aldehyde/alkyne, 1,2-diol/azide, 1,2-diol/alkyne, 1,2-diol/aniline, 1,2-aminoalcohol/azide, 1,2-aminoalcohol/alkyne, 1,2-aminoalcohol/aniline, O-alkylated hydroxylamine/azide, O-alkylated hydroxylamine/alkyne, alkylated hydrazine/azide, alkylated hydrazine/alkyne, acylated hydrazine/azide, acylated hydrazine/alkyne, azide/aniline, alkyne/aniline,
a combination of ketone with a chemical group comprising 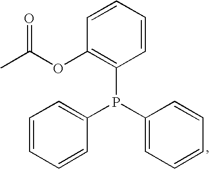
a combination of ketone with a chemical group comprising 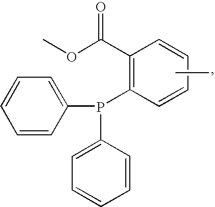
a combination of aldehyde with a chemical group comprising 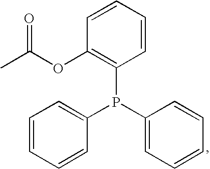
a combination of aldehyde with a chemical group comprising 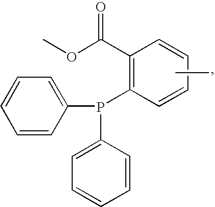
a combination of 1,2-diol with a chemical group comprising 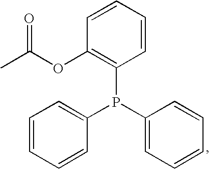
a combination of 1,2-diol with a chemical group comprising 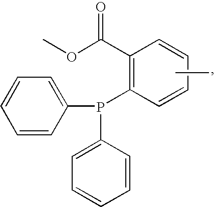
a combination of 1,2-aminoalcohol with a chemical group comprising 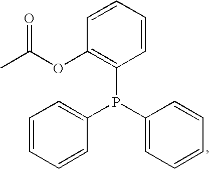
a combination of 1,2-aminoalcohol with a chemical group comprising 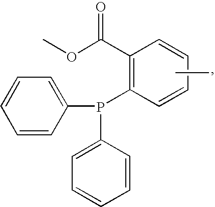
a combination of aniline with a chemical group comprising 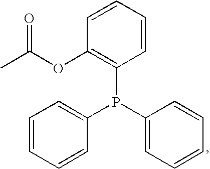
or a combination of aniline with a chemical group comprising [chemical structure]
